(19) United States
(12) Patent Application Publication
(10) Pub. No.: US 2026/0007733 A1
(43) Pub. Date: Jan. 8, 2026

(54) Specific Saccharide Fragment for Development of Vibrio Cholerae Vaccines

(71) Applicant: Jiangnan University, Wuxi (CN)

(72) Inventors: Jing HU, Wuxi (CN); Jian YIN, Wuxi (CN); Guodong CHEN, Wuxi (CN)

(21) Appl. No.: 19/258,844

(22) Filed: Jul. 2, 2025

Related U.S. Application Data
(63) Continuation of application No. PCT/CN2024/127370, filed on Oct. 25, 2024.

(30) Foreign Application Priority Data
Jun. 19, 2024 (CN) 2024107916722

(51) Int. Cl. A61K 39/02; A61K 39/00; A61K 39/385; G01N 33/569

(52) U.S. Cl. CPC A61K 39/107; A61K 39/385; G01N 33/56911; A61K 2039/6031; G01N 2333/28

(57) ABSTRACT

The present disclosure discloses a specific saccharide fragment for development of Vibrio cholerae vaccines, and belongs to the field of medicine. In the present disclosure, a saccharide fragment related to a trisaccharide of V. cholerae O100 serotype O-antigen is chemically synthesized. Combined with a glycan microarray technology, the structure-activity relationship between different saccharide fragments and antigenicity thereof is evaluated at a molecular level. Glycan microarray screening indicates that 3-hydroxybutyryl is an essential structural feature of the O-antigen. A non-reducing end disaccharide carrying 3-hydroxybutyryl is a potential minimal antigenic epitope, and the disaccharide has strong binding capacity to antibodies and a simple structure, and may serves as a specific saccharide fragment for vaccine development. A glycoconjugate vaccine designed based on the specific saccharide fragment may solve the challenges of difficulty in culturing pathogenic bacteria and heterogeneity of saccharide antigens in naturally extracted polysaccharide vaccines. The present disclosure has bright application prospects in the development of glycoconjugate V. cholerae vaccine, infection detection, and new drug development.

### TECHNICAL FIELD

The present disclosure relates to development of a specific saccharide fragment for development of Vibrio cholerae vaccines, and belongs to the field of medicine.

### BACKGROUND

Cholera is a serious and life-threatening acute intestinal infectious disease caused by Vibrio cholerae, and is legally classified as a category A infectious disease in China. According to the World Health Organization, 30 countries reported cases of cholera in 2023, and cholera causes over one million cases of diarrhea and a large number of deaths worldwide each year.

V. cholerae typically persists in aquatic environments and reproduces in the human gut. This high-risk infection can cause watery diarrhea within hours, accompanied by dehydration, vomiting, coma, and even death.

According to different cell surface lipopolysaccharide (LPS)O-antigens, V. cholerae is classified into over 200 serotypes. Due to severe drug resistance of V. cholerae and shortage of oral vaccines, there is an urgent need to provide a new vaccine against cholera. In recent years, glycoconjugate vaccines have shown promising prospects, especially for the protection of young children. Development of a multivalent glycoconjugate vaccine that covers all important serotypes of cholera are considered an important technological direction for eradicating cholera.

### SUMMARY

### Technical Problem

In view of the shortcomings of the prior art, the present disclosure relates to development of a specific saccharide fragment for development of Vibrio cholerae vaccines.

### Technical Solution

In the present disclosure, based on chemical synthesis, a saccharide fragment related to a trisaccharide repeating unit of V. cholerae O100 serotype O-antigen is obtained. The synthesized saccharide fragment is immobilized on a surface of a microarray to prepare a glycan microarray. Subsequently, the glycan microarray is incubated with anti-sera to specifically recognize the saccharide fragment by IgG antibodies in the anti-sera. Subsequently, antibodies in the glycan microarray are labeled with fluorescently labeled secondary antibodies, and a specific saccharide fragment that can be used for the development of V. cholerae vaccine is obtained via fluorescence scanning and quantitative analysis.

A first objective of the present disclosure is to provide a specific saccharide fragment for development of V. cholerae vaccine, having a structure of R₂—[U1]ₐ-[U2]—[U3]_{b}-O-Linker. The structures of U1, U2, and U3 are as follows:

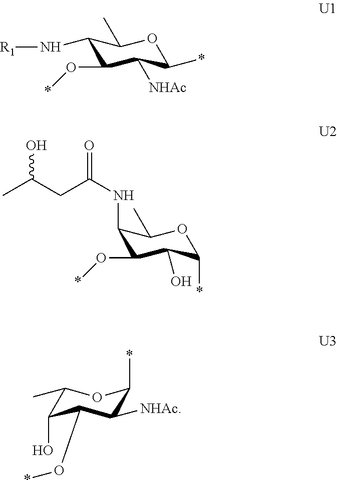

In formula I, a and b represent the quantities of U1 (D-quinosamine) and U3 (L-fucoidamine), respectively, where a and b are 0 or 1, respectively; R₁ represents one of 3,5-dihydroxyhexanoyl or acetyl groups; R₂ represents H (hydrogen), or H-U3-(monosaccharide), or H-U2-U3-(disaccharide), or H-U1-U2-U3-(trisaccharide); and Linker represents-(CH₂)ₙ—NH₂ or —(CH)ₙSH, n=2−40.

In an implementation of the present disclosure, a group at position 4 of monosaccharide U2 in the specific saccharide fragment is (R)-3-hydroxybutyrylamino or(S)-3-hydroxybutyrylamino.

In an implementation of the present disclosure, an (R)-3-hydroxybutyryl modifying group is a key constituent of the specific saccharide fragment.

In an implementation of the present disclosure, the specific saccharide fragment is further selected from:

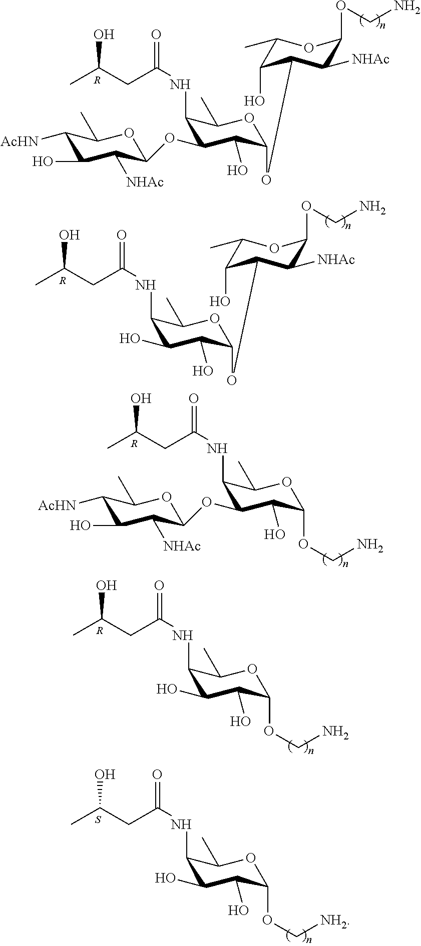

In an implementation of the present disclosure, n=2−40, further specifically, optionally 5.

In an implementation of the present disclosure, the synthesis of the saccharide fragment in a glycan library is achieved via a series of reduction acylation, amide condensation, catalytic hydrogenation, and the like, using three monosaccharide building blocks and five carboxylic acid derivatives.

In an implementation of the present disclosure, a reducing agent used for the reduction acylation is one of zinc powder, triphenylphosphine, 1,3-propanedithiol, lithium aluminum hydride, trimethylphosphine, stannous chloride dihydrate, sodium borohydride, and sodium cyanoborohydride.

In an implementation of the present disclosure, a condensing agent used for the amide condensation is one of dicyclohexylcarbodiimide (DCC), diisopropylcarbodiimide (DIC), 1-(3-dimethylaminopropyl)-3-ethylcarbodiimide (EDC), diphenylphosphoryl azide (DPPA), diphenylphosphinyl chloride (DPPCI), diphenyl cyanophosphonate (DECP), 2-(7-azabenzotriazole)-N,N,N′,N′-tetramethylurea hexafluorophosphate (HATU), benzotriazole-N,N,N′,N′-tetramethylurea hexafluorophosphate (HBTU), and 6-chlorobenzotriazole-1,1,3,3-tetramethylurea hexafluorophosphate (HCTU).

In an implementation of the present disclosure, a catalyst used for the catalytic hydrogenation may be a 10% palladium on carbon catalyst, palladium hydroxide, or the like.

In an implementation of the present disclosure, a solvent used for the catalytic hydrogenation may be one of a water/methanol/dichloromethane/acetic acid mixture, a water/tert-butanol/dichloromethane mixture, a water/tert-butanol/ethyl acetate mixture, and a water/tert-butanol/tetrahydrofuran mixture.

In an implementation of the present disclosure, the catalytic hydrogenation may be conducted at a reaction temperature of 0-40° C.

The present disclosure further provides an application of the specific saccharide fragment in preparation of V. cholerae vaccine.

The present disclosure further provides an application of the specific saccharide fragment in preparation of drugs for prevention or treatment of V. cholerae infection.

The present disclosure further provides a pharmaceutical composition containing the specific saccharide fragment and pharmaceutical excipients.

The present disclosure further provides a pharmaceutical composition containing any one or a combination of more of the five types of specific saccharide fragments, and pharmaceutical excipients.

In a specific implementation of the present disclosure, the specific saccharide fragment is preferably

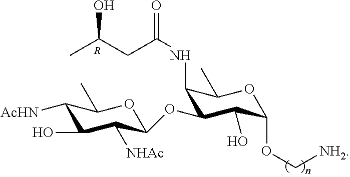

In an implementation of the present disclosure, the pharmaceutical excipients include a solvent, an osmotic pressure regulator, a stabilizer, a preservative, and a pH regulator.

The present disclosure further provides a method for preparing the glycan microarray, including binding a Linker structure of the specific saccharide fragment with the glycan microarray.

In a specific implementation of the present disclosure, the method for preparing the glycan microarray specifically includes the following steps:

Step 1: The obtained sugar fragment is dissolved in a 50 mM phosphate solution (pH=8.5), printed onto the microarray using a microarray spotter, and incubated at room temperature and 65% humidity overnight to covalently bind the saccharide fragment to the microarray; and after incubation, the microarray is quenched by treatment with a mixed solution of 100 nM ethanolamine and 50 nM sodium phosphate solution (pH=9) at 50° C. for 1 h.

Step 2: The glycan microarray is probed with diluted rabbit anti-sera and co-incubated to specifically bind IgG antibodies in the anti-sera to the saccharide fragment, and unbound serum antibodies are washed away; and subsequently, fluorescently labeled secondary antibodies (anti-IgG antibodies) are used to bind the IgG antibodies on the microarray and incubated, and unbound secondary antibodies are washed away.

Step 3: Fluorescence scanning is carried out on a microarray scanner, and based on a scanning result, a saccharide fragment with good antigenicity, i.e., the specific saccharide fragment is screened out.

In a specific implementation of the present disclosure, the saccharide fragment may be diluted at a phosphate concentration of 10-100 mM, preferably 50 mM, and a pH of 8-9, preferably 8.5.

In a specific implementation of the present disclosure, the concentration of the specific saccharide fragment is 0.01-10 mM, preferably 0.05 mM.

In a specific implementation of the present disclosure, the incubation is carried out at room temperature (20-30° C.), and a humidity of 50-70%, preferably 65%.

In a specific implementation of the present disclosure, the sera are diluted at a ratio of 1:10-1:500, preferably 1:200.

In a specific implementation of the present disclosure, the fluorescently labeled secondary antibodies are Cy3 labeled goat anti-human or goat anti-rabbit IgG antibodies, with a dilution ratio of 1:30-1:1,000, preferably 1:400.

The present disclosure further provides an apparatus for detecting V. cholerae O100 infection, and the apparatus includes a microarray, a specific saccharide fragment, a dilution solution of a test sample, and fluorescently labeled secondary antibodies.

In an implementation of the present disclosure, after immobilization and quenching of the specific saccharide fragment are carried out, the apparatus for detecting infection may be partitioned into multiple micro-wells using molds of different specifications, allowing for simultaneous detection of a plurality of samples conveniently and efficiently.

In a specific implementation of the present disclosure, the molds may be 16-cell, 64-cell, or 128-cell molds.

In a specific implementation of the present disclosure, after immobilization and quenching of the specific saccharide fragment, the specific saccharide fragment may be retained for future use, and a surface of a microarray may be directly probed with diluted sera more conveniently and efficiently when in use.

The present disclosure further provides a V. cholerae glycoprotein conjugate for vaccine development, and the conjugate is obtained by conjugating a Linker structure of the specific saccharide fragment with a protein.

In an implementation of the present disclosure, a carrier protein used in the glycoprotein conjugate includes one of a diphtheria toxin mutant protein (CRM197), keyhole limpet hemocyanin (KLH), bovine serum albumin (BSA), meningococcal outer-membrane protein complex (OMPC), tetanus toxoid (TT), or diphtheria toxoid (DT).

### Beneficial Effects:

Glycoconjugate vaccines based on naturally extracted polysaccharide antigens in the prior art have many drawbacks, such as difficulty in cultivating some pathogenic bacteria, failure to obtain a sufficient quantity of extracts, and suspectable contamination with impurities. In contrast, a glycoconjugate vaccine based on synthetic oligosaccharide antigens in the present disclosure not only avoids the mixing of virulence factors of pathogenic bacteria, but also obtains the smallest effective antigen epitope. In addition, application of the structurally well-defined specific oligosaccharide antigens also makes production of saccharide vaccines more repetitive.

The present disclosure constructs a glycan library via chemical synthesis and employs a glycan microarray technology to provide the specific saccharide fragment for the development of the V. cholerae vaccine. A screening result using the glycan microarray indicates that a monosaccharide, a disaccharide, and a trisaccharide modified with a 3-hydroxyhexanoyl group may be specifically recognized by the anti-sera immunized with the lipopolysaccharide of the V. cholerae O100 serotype. A monosaccharide, a disaccharide, and a trisaccharide without modification of the 3-hydroxyhexanoyl group cannot be recognized, indicating that 3-hydroxybutyryl modification is a typical feature of the O-antigen and a key modification for the design of synthetic glycoconjugate V. cholerae vaccine. A non-reducing end disaccharide

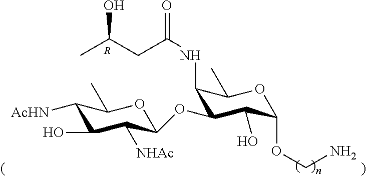

has a simple structure and good antigenicity, is considered the potential minimal antigenic epitope, and may be used as a specific saccharide fragment for the development of V. cholerae vaccine. In addition, the present disclosure further provides theoretical basis for the detection of V. cholerae infection, development of new drugs, and the like.

### BRIEF DESCRIPTION OF FIGURES

FIG. 1 shows a schematic diagram of a method according to the present disclosure.

FIG. 2 shows structures of 11 saccharide fragments in a glycan library of the present disclosure.

FIG. 3 shows a synthetic route of a compound 10.

FIG. 4 shows synthetic routes of compounds 5, 6, 8, 9, and 11.

FIG. 5 shows synthetic routes of compounds 2 and 3.

FIG. 6 shows synthetic routes of compounds 1, 4 and 7.

FIG. 7A shows 1H-NMR spectra of Vibrio cholerae O100 serotype lipopolysaccharide O-antigen, and FIG. 7B shows 13C-NMR spectra of Vibrio cholerae O100 serotype lipopolysaccharide O-antigen.

FIG. 8 shows IgG antibody titers in rabbit sera detected via ELISA.

FIG. 9 shows a screening result using a glycan microarray, including: a schematic structural diagram of 11 saccharide fragments; a microarray printing pattern; a microarray scan result; and quantification of the mean fluorescence intensity. Error bars represent the standard error of the mean of two spots at uniform concentration.

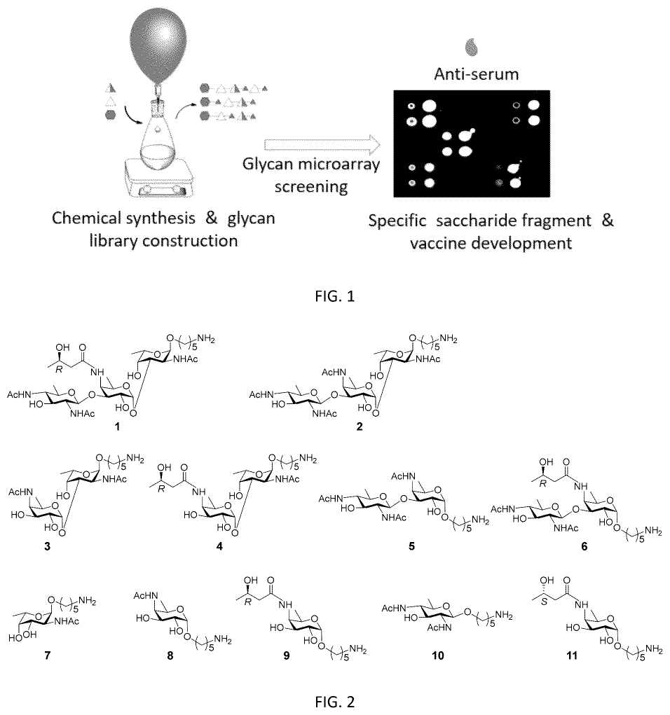

### DETAILED DESCRIPTION

Commercial agents used in experiments are used as received without further treatment, and anhydrous solvents used in reactions are prepared by an MBraun MB-SPS 800 type solvent drying system. Solvents used for silica gel column chromatography are all analytically pure and are used after reduced pressure distillation. A silica gel plate used for thin layer chromatography (TLC) is a glass-based or aluminum foil-based silica gel plate prepared from 60-F254 silica gel, and the silica gel used for normal-phase silica gel column chromatography is 200-300 mesh silica gel.

The yield of each reaction step is calculated as follows: (amount of target product substance/amount of raw material substance)*100%. The structures of products are identified via nuclear magnetic resonance (NMR) spectra, infrared spectra, optical rotation, and high-resolution mass spectra. The purity of products is analyzed by NMR spectra. A proton NMR spectrum, a carbon-13 NMR spectrum, and two-dimensional NMR spectrum are measured by Bruker Ascend 600 M and 400 M NMR spectrometers at 25° C. High-resolution mass spectra are obtained by an Agilent 6220 electrospray ionization time-of-flight mass spectrometer. Infrared spectra are obtained by a Thermo Fisher Scientific Nicolet iS5 FT-IR spectrometer. The optical rotation is measured by a Schmidt & Haensch UniPol L 10000 fully-automatic polarimeter at 589 nm. The unit of a measured concentration (c) is g/100 mL.

In this specification, certain implementations may be disclosed in a format that falls within a certain range. It should be understood that the description of “being within a certain range” is only for convenience and conciseness, and should not be interpreted as a rigid limitation on the disclosed scope. Although the present disclosure has been disclosed above with preferred examples, they are not intended to limit the present disclosure. Anyone familiar with the technology can make various changes and modifications without departing from the principle and scope of the present disclosure. Therefore, the protection scope of the present disclosure should be defined by claims.

### Example 1

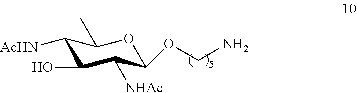

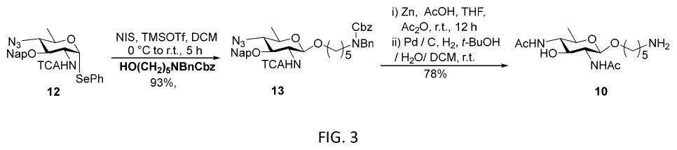

Compound 10 was synthesized, as shown in FIG. 3.

Compound 12 (selenoglycoside, self-made by reference to Codée. et al, Organic & Biomolecular Chemistry 2020,18 (15), 2834-2837) and N-benzyl-N-benzyloxycarbonyl-5-aminopentanol underwent a glycosylation reaction in the presence of trimethylsilyl trifluoromethanesulfonate (TMSOTf) and N-iodosuccinimide (NIS), resulting in a compound 13. Subsequently, zinc powder, acetic acid, and acetic anhydride were used for reductive acylation, followed by catalytic hydrogenation to obtain deprotected target compound 10.

### Specific Experimental Operations and Steps:

Compound 13: Selenoglycoside 12 (170 mg, 0.28 mmol) and N-benzyl-N-benzyloxycarbonyl-5-aminopentanol (136 mg, 0.42 mmol) were mixed, azeotropically evaporated with toluene (3×15 ml), and spin dried. Then a newly activated 4 Å molecular sieve was added, and dried under vacuum for 2 h using an oil pump. Subsequently, the resulting mixture was dissolved in a DCM (10 mL) solution, and NIS (94 mg, 0.42 mmol) and TMSOTf (20 μl, 0.11 mmol) were added slowly at 0° C. After being stirred for 4 h, the reaction mixture was neutralized with one drop of Et₃N at 0° C. and heated to room temperature, and the 4 Å molecular sieve was filtered out. A filtrate was washed with a 10% Na₂S₂O₃ aqueous solution, a saturated NaHCO₃ aqueous solution, and saturated brine. A combined organic layer was dried over Na₂SO₄, filtered, and evaporated under vacuum. The crude product was purified by silica gel column chromatography (petroleum ether::ethyl acetate=3:1) to obtain compound 13 (202 mg, 0.26 mmol, 93%). [α]²⁵_{D}=+30.2° (c=0.47, CHCl₃); IR vmax (film) 3029, 2943, 2872, 2109, 1697, 1525, 1423, 1361, 1230, 1082, 821, 758, 698 cm⁻¹; ¹H NMR (400 MHZ, Chloroform-d) δ 7.88-7.79 (m, 4H, Ar), 7.50 (d, J=7.6 Hz, 3H, Ar), 7.43-7.11 (m, 11H, Ar), 5.18 (d, J=20.4 Hz, 2H, Ar—CH₂), 5.01 (d, J=10.8 Hz, 1H, Ar—CH₂), 4.88 (d, J=10.1 Hz, 2H, 1-H, Ar—CH₂), 4.50 (s, 2H, Ar—CH₂), 4.22 (dt, J=22.3, 9.7 Hz, 1H, 3-H), 3.87 (s, 1H, linker-OCH₂), 3.53-3.33 (m, 3H, linker-OCH₂, 2-H, 5-H), 3.32-3.10 (m, 3H,4-H, linker-NCH₂), 1.58 (s, 4H, linker-CH₂), 1.41 (d, J=6.1 Hz, 3H, 6-H), 1.38-1.23 (m, 2H, linker-CH₂). ¹³C NMR (101 MHZ, Chloroform-d) δ 162.1 (NHAc-C═O) 156.7 (Cbz-C═O) 137.9 (Ar), 133.2 (Ar), 128.6 (Ar), 128.5 (Ar), 128.4 (Ar), 128.0 (Ar), 127.95 (Ar), 127.8 (Ar), 127.7 (Ar), 127.3 (Ar), 127.2 (Ar), 127.1 (Ar), 126.2 (Ar), 126.0 (Ar), 98.6 (1-H), 78.4 (3-H), 75.2 (Ar—CH₂), 70.7 (5-H), 70.0 (linker-OCH₂), 68.9 (4-C), 67.2 (Ar—CH₂), 59.6 (2-C), 50.3 (Ar—CH₂), 47.2 (linker-NCH₂), 29.2 (linker-CH₂), 28.8 (linker-CH₂), 23.5 (linker-CH₂), 18.5 (6-C). HR-ESI-MS (m/z): calcd for C₃₉H₄₂O₆N₅Cl₃Na⁺ (M+Na)⁺: 804.2093 found: 804.210.

Compound 10: Compound 13 (50 mg, 64 umol) was dissolved in THF/Ac₂O/AcOH (3/2/1, v/v/v, 3 mL), and freshly activated Zn (1 g) was added. After being stirred overnight at room temperature, the mixture was diluted and filtered. A filtrate was washed with a saturated NaHCO₃ solution and a saturated sodium chloride solution. An organic layer was dried over anhydrous Na₂SO₄, filtered, and evaporated under vacuum, and then dried under vacuum using an oil pump for 2 h. Subsequently, the organic layer was dissolved in DCM/t-BuOH/H₂O (2/1/1, v/v/v, 2 mL), and 10% Pd/C (50 mg) was added. After being stirred in hydrogen (4 atm) for 36 h, the mixture was filtered and washed with water. Subsequently, a residue was purified using a Sep-Pak column C18 (Macherey-Nagel, Düren, Germany) with water and methanol as eluents to obtain compound 10 (13.8 mg, 41.6 μmol, two-step yield: 65%). ¹H NMR (600 MHZ, Deuterium Oxide) δ 4.37 (d, J=8.5 Hz, 1H, 1-H), 3.76 (dt, J=11.5, 6.2 Hz, 1H, linker-OCH₂), 3.58 (t, J=9.0 Hz, 1H, 2-H), 3.51-3.42 (m, 4H, 3-H, 4-H, 5-H, linker-OCH₂), 2.87 (t, J=7.7 Hz, 2H, linker-NCH₂), 1.92 (d, J=3.3 Hz, 6H, NHAc), 1.55 (m, J=7.7 Hz, linker-CH₂), 1.48 (p, J=6.7 Hz, 2H, linker-CH₂), 1.28 (dt, J=9.1, 5.5 Hz, 2H, linker-CH₂), 1.11 (d, J=5.0 Hz, 3H, 6-CH₃). ¹³C NMR (151 MHZ, Deuterium Oxide) δ 174.6 (NHAc-C═O), 174.5 (NHAc-C═O), 101.0 (1-C), 71.6, 71.0, 70.1 (linker-OCH₂), 57.1, 56.3 (2-C), 39.3 (linker-NCH₂), 28.1 (linker-CH₂), 26.4 (linker-CH₂), 22.2 (NHAc-CH₃, linker-CH₂), 16.8 (6-CH₃). HR-ESI-MS (m/z): calcd for C₁₅H₃₀O₅N₃⁺ (M+H)⁺: 332.2180 found: 332.2219.

### Example 2

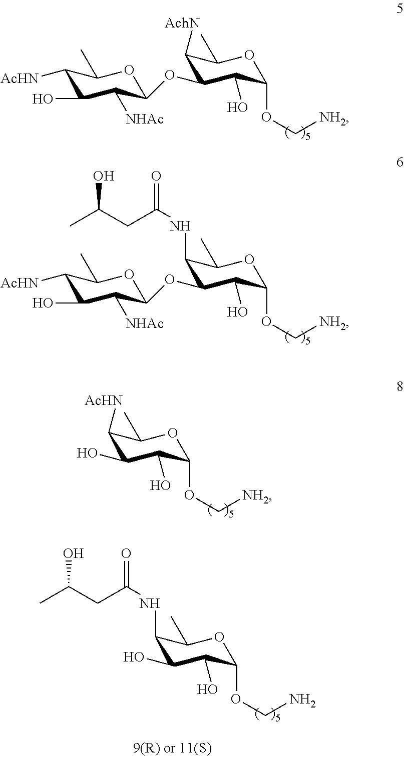

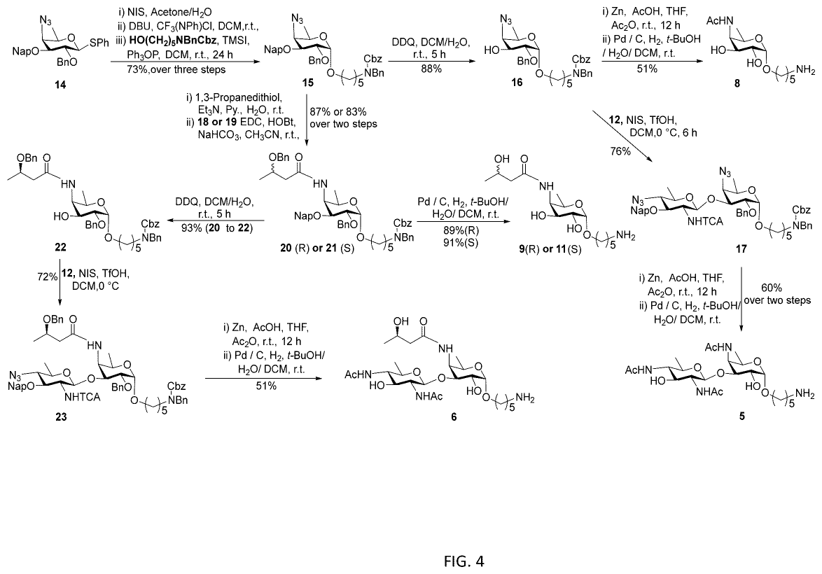

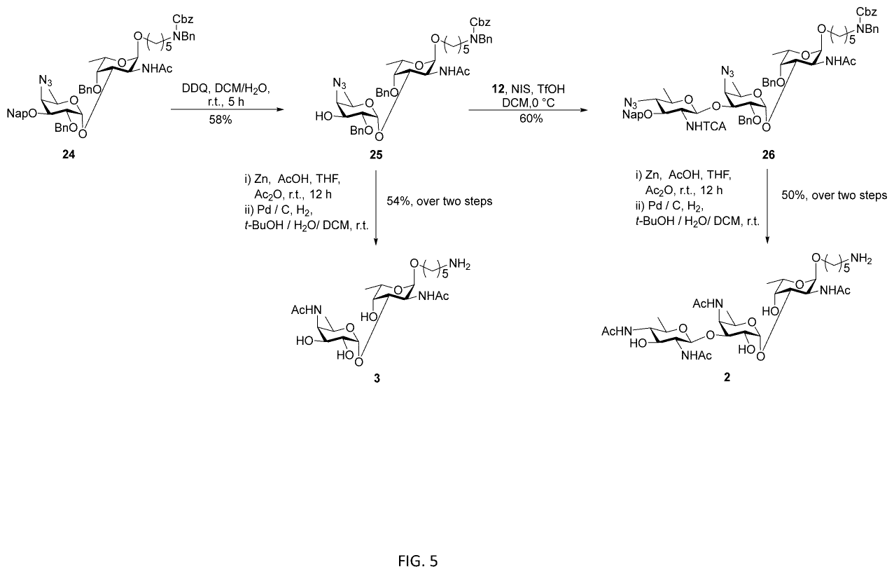

Compounds 5, 6, 8, 9, and 11 were synthesized, as shown in FIG. 4.

Compound 14 (self-made by reference to doctoral thesis of Cai Juntao, Jiangnan University, 2020) was hydrolyzed with NIS, esterified with trifluoro-N-phenylacetimidoyl chloride, and then dissolved in a DCM solution in the presence of triphenylphosphine oxide (Ph₃OP) and trimethylsilyl iodide (TMSI) to obtain compound 15. 2-Naphthalene methylene was removed by 2,3-dichloro-5,6-dicyano-1,4-benzoquinone (DDQ) to obtain acceptor 16. Acceptor 16 and the donor selenoglycoside 12 underwent a glycosylation reaction to obtain disaccharide 17. Disaccharide 17 and compound 16 were reduced and acylated with zinc powder and acetic anhydride, respectively, followed by direct deprotection to synthesize compounds 5 and 8 respectively. 1,3-Propanedithiol was used to reduce an azide group in compound 15, and amide condensation was carried out with butyric acids 18 and 19 (self-made by reference to Tanasova. et al., Angew. Chem. Int. Ed. 2015, 54 (14), 4274-4278), respectively in the presence of 1-(3-dimethylaminopropyl)-3-ethylcarbodiimide hydrochloride (EDC) and 1-hydroxybenzotriazole (HOBt) to obtain compounds 20 and 21. Compounds 20 and 21 were deprotected respectively to obtain compounds 9 and 11. 2-Naphthalene methylene was selectively removed from compound 20 to obtain acceptor 22. Acceptor 22 and the donor selenoglycoside 12 underwent a glycosylation reaction to obtain disaccharide 23, followed by deprotection to obtain compound 6.

### Specific Experimental Operations and Steps:

Compound 15: Compound 14 (200 mg, 0.38 mmol) was dissolved in acetone and H₂O (10:1, v/v, 5.5 mL) at room temperature and stirred uniformly. Then NIS (171.4 mg, 0.7 mmol) was added and stirred for 1 h. After TLC indicated that the reaction was complete, the mixture was diluted with ethyl acetate and washed with 10% (w/v) Na₂S₂O₃. An organic layer was dried over Na₂SO₄, filtered and concentrated under vacuum. A residue was purified by silica gel column chromatography (petroleum ether/ethyl acetate=20/1 to 1/1, v/v) to obtain a compound intermediate. The compound intermediate was dissolved in a DCM solution (4.8 mL) at 0° C., and then 2,2,2-trifluoro-N-phenylacetimidoyl chloride (171 μL, 1.14 mmol) and 1,8-diazabicycloundec-7-ene (DBU) (171 μL, 1.14 mmol) were added. A reaction was carried out at 0° C. for 3 h, the mixture was concentrated under vacuum, and then purified by silica gel column chromatography (petroleum ether/ethyl acetate=20/1 to 5/1, v/v) to obtain trifluoroacetamide ester. The trifluoroacetamide ester and N-benzyl-N-benzyloxycarbonyl-5-aminopentanol (187.3 mg, 0.572 mmol) were co-evaporated with toluene three times, and then dissolved in anhydrous DCM (1 mL). A preactivated dry molecular sieve 4 Å and Ph₃OP (848 mg, 3.05 mmol) were added. Then TMSI (56.5 μL, 0.38 mmol, 1.0 eq) was slowly added to the mixture. The mixture was stirred for reaction at room temperature until TLC analysis indicates complete reaction. The solution was diluted, and the reaction was quenched with saturated Na₂S₂O₃. The organic phase was washed with water and brine, dried over anhydrous Na₂SO₄, filtered, concentrated under vacuum, and purified by silica gel column chromatography (petroleum ether/ethyl acetate=20/1 to 1/1, v/v) to obtain compound 15 (203 mg, 0.28 mmol, three-step yield: 73%). [α]²⁵_{D}=+11.1° (c=1.0, CHCl₃); IR vmax (film) 3029, 2944, 2903, 2108, 1697, 1454, 1361, 1279, 1227, 1127, 1096, 1044, 820, 755, 698 cm⁻¹; ¹H NMR (400 MHZ, Chloroform-d) δ 7.87-7.76 (m, 4H, Ar), 7.49 (ddd, J=16.3, 7.5, 2.5 Hz, 3H, Ar), 7.35-7.14 (m, 17H, Ar), 5.17 (d, J=9.5 Hz, 2H, Ar—CH₂), 5.00 (d, J=11.9 Hz, 1H, Ar—CH₂), 4.88 (d, J=11.9 Hz, 1H, Ar—CH₂), 4.81 (d, J=12.0 Hz, 1H, Ar—CH₂), 4.70-4.60 (m, 2H, Ar—CH₂, 1-H), 4.48 (d, J=8.0 Hz, 2H, Ar—CH₂), 4.05 (s, 1H, 3-H), 3.85 (dd, J=9.8, 3.8 Hz, 2H, 5-H, 2-H), 3.71 (s, 1H, 4-H), 3.51 (s, 1H, Linker-OCH₂), 3.34 (s, 1H, Linker-OCH₂), 3.27-3.14 (m, 2H, Linker-NCH₂), 1.57 (m, 4H, Linker-CH₂) 1.25 (m, 2H, Linker-CH₂), 1.19 (d, J=6.4 Hz, 3H, 6-CH₃). ¹³C NMR (101 MHZ, Chloroform-d) δ 138.4 (Ar), 135.8 (Ar), 133.3 (Ar), 133.0 (Ar), 128.6 (Ar), 128.5 (Ar), 128.4 (Ar), 128.2 (Ar), 128.0 (Ar), 127.9 (Ar), 127.8 (Ar), 127.77 V, 127.7 (Ar), 127.2 (Ar), 126.4 (Ar), 126.1 (Ar), 125.9 (Ar), 125.7 (Ar), 97.4 (1-H), 78.1 (3-H), 76.1 (2-H), 73.5 (Ar—CH₂), 73.3 (Ar—CH₂), 68.2 (Ar—CH₂), 67.2 (Ar—CH₂), 65.2 (4-H), 64.3 (5-H), 50.2 (Ar—CH₂) 29.1 (Linker-CH₂), 23.5 (Linker-CH₂), 17.3 (6-CH₃). HR-ESI-MS (m/z): calcd for C₄₄H₄₈O₆N₄Na⁺ (M+Na)⁺: 751.3466 found: 751.3501.

Compound 16: H₂O (1 mL) was added to a solution of compound 15 (16.75 mg, 23.0 μmol) in DCM (2.0 mL), followed by addition of DDQ (7.7 mg, 35.0 μmol). The reaction mixture was stirred at room temperature for 5 h, after which TLC indicated that the reaction was complete. The mixture was diluted with DCM (2×10 mL) and washed with saturated NaHCO₃ (20 mL). An organic layer was dried over anhydrous Na₂SO₄, and the reaction mixture was concentrated under vacuum. A residue was purified by silica gel column chromatography (petroleum ether: acetone=1:1) to obtain compound 16 (11.9 mg, 20.2 μmol, 88%). [α]²⁵_{D}=+30.8° (c=0.3, CHCl₃); IR vmax (film) 3029, 2944, 2903, 2108, 1697, 1454, 1422, 1361, 1279, 1227, 1127, 1096, 1044, 820,755, 698 cm⁻¹; ¹H NMR (400 MHZ, Chloroform-d) δ 7.44-7.10 (m, 17H, Ar), 5.17 (d, J=10.3 Hz, 2H, Ar—CH₂), 4.74-4.56 (m, 3H, Ar—CH₂, 1-H), 4.48 (d, J=7.5 Hz, 2H, Ar—CH₂), 4.17 (s, 1H, 3-H), 3.94 (s, 1H, 5-H), 3.77-3.65 (m, 2H, 4-H, 2-H), 3.53 (s, 1H, Linker- OCH₂), 3.31-3.12 (m, 3H, Linker-OCH₂, Linker-NCH₂), 2.52 (s, 1H, 3-OH), 1.51 (m, 4H, Linker-CH₂) 1.23 (d, J=6.5 Hz, 5H, Linker-CH₂, 6-CH₃). ¹³C NMR (101 MHZ, Chloroform-d) δ 137.9 (Ar), 137.8 (Ar), 128.6 (Ar), 128.6 (Ar), 128.5 (Ar), 128.2 (Ar), 128.1 (Ar), 128.0 (Ar), 127.8 (Ar), 127.3 (Ar), 96.5 (1-C), 77.0 (2-C), 72.7 (Ar—CH₂), 69.9 (3-H), 68.2 (Ar—CH₂), 67.2 (Ar—CH₂), 65.9 (4-C), 64.6 (5-C), 50.3 (Ar—CH₂), 47.1 (Linker-OCH₂), 29.2 (Linker-CH₂), 23.5 (Linker-CH₂), 17.3 (6-CH₃). HR-ESI-MS (m/z): calcd for C₃₃H₄₀O₆N₄Na⁺ (M+Na)⁺: 611.2840 found: 611.2895.

Compound 17: Selenoglycoside 12 (49 mg, 0.08 mmol) and acceptor 16 (56.5 mg, 0.10 mmol) were mixed, azeotropically evaporated with toluene (3×5 mL) to remove water, and dried under vacuum using an oil pump for 2 h. The donor-acceptor mixture was dissolved in DCM (8 mL) at 0° C., a freshly activated 4 Å molecular sieve was added, and then NIS (27 mg, 0.12 mmol) and TfOH (14.2 μL, 0.16 mmol) were slowly added. After stirring for 4 h, a drop of triethylamine (Et₃N) was added to the reaction system for neutralization, and the 4 Å molecular sieve was filtered. A filtrate was washed with a 10% Na₂S₂O₃ solution and a saturated NaHCO₃ solution, respectively. A combined organic layer was dried over anhydrous Na₂SO₄, filtered, and concentrated. A crude product was purified by silica gel column chromatography (petroleum ether:ethyl acetate=2:1) to obtain compound 17 (63.6 mg, 0.061 mmol, 76% yield). [α]²⁵_{D}=+43.7° (c=0.6, CHCl₃); IR vmax (film) 3032, 2947, 2903, 2108, 1678, 1454, 1422, 1361, 1279, 1226, 1127, 1096, 1044, 820,755, 698 cm⁻¹; ¹H NMR (600 MHZ, Chloroform-d) δ 7.81 (dd, J=10.7, 8.3 Hz, 4H, Ar), 7.47 (dd, J=8.7, 5.0 Hz, 3H, Ar), 7.40-7.13 (m, 17H, Ar), 6.99 (d, J=7.8 Hz, 1H, NHAc-H), 5.23 (d, J=8.2 Hz, 1H, 1′-H), 5.18 (d, J=18.2 Hz, 2H, Ar—CH₂), 4.97 (d, J=10.8 Hz, 1H, Ar—CH₂), 4.86 (d, J=10.7 Hz, 1H, Ar—CH₂), 4.77 (d, J=12.2 Hz, 1H, Ar—CH₂), 4.53-4.38 (m, 4H, 1-H, Ar—CH₂), 4.23 (m, 1H, 3-H), 4.09 (m, 1H, 3′-H), 3.91 (m, 1H, 5-H), 3.81 (d, J=7.9 Hz, 1H, 4-H), 3.74 (dd, J=10.2, 3.7 Hz, 1H, 2-H), 3.58 (dt, J=18.0, 9.0 Hz, 1H, 2′-H), 3.41 (d, J=23.7 Hz, 2H, 5′-H, Linker-OCH₂), 3.30-3.10 (m, 4H, 4′-H Linker-OCH₂, Linker-NCH₂), 1.51 (dt, J=27.9, 6.9 Hz, 4H, Linker-CH₂), 1.39 (d, J=5.6 Hz, 3H, 6′-H), 1.26 (d, J=10.0 Hz, 2H, Linker-CH₂), 1.17 (d, J=6.2 Hz, 3H, 6-CH₃). ¹³C NMR (151 MHZ, Chloroform-d) δ 161.7 (NH—C═O), 138.5 (Ar), 137.9 (Ar), 134.7 (Ar), 133.3 (Ar), 133.1 (Ar), 128.6 (Ar), 128.5 (Ar), 128.3 (Ar), 128.0 (Ar), 127.97 (Ar), 127.94 (Ar), 127.8 (Ar), 127.7 (Ar), 127.4 (Ar), 127.2 (Ar), 127.0 (Ar), 126.1 (Ar), 126.1 (Ar), 125.9 (Ar), 99.1 (1′-H), 97.0 (1-H), 78.6 (3′-C), 76.4, 76.3 (3-C, 2-C), 75.1 (Ar—CH₂), 73.3 (Ar—CH₂), 70.8 (5′-H), 68.7 (4′-H), 68.2 (Linker-OCH₂), 67.2 (Ar—CH₂), 66.5 (4-C), 63.9 (5-C), 59.6 (2′-H), 50.3 (Ar—CH₂), 46.1 (Linker-NCH₂), 29.1 (Linker-CH₂), 27.5 (Linker-CH₂), 23.4 (Linker-CH₂), 18.5 (6′-C), 17.2 (6-C). HR-ESI-MS (m/z): calcd for C₅₂H₅₇O₉N₈Cl₃Na⁺ (M+Na)⁺: 1065.3206 found: 1065.3227.

Compound 5: Compound 17 (40 mg, 38.38 μmol) was dissolved in a mixed solution of THF/Ac₂O/AcOH (3/2/1, v/v/v, 3 mL) and freshly activated Zn (1 g) was added. After being stirred overnight at room temperature, the mixture was diluted with DCM and filtered. A filtrate was washed with a saturated NaHCO₃ aqueous solution and saturated brine. A combined organic layer was dried over anhydrous Na₂SO₄, filtered, evaporated under vacuum, and purified by silica gel column chromatography (DCM/MeOH=50/1 to 10/1, v/v) to obtain a compound intermediate. Then the compound intermediate was dissolved in a DCM/t-BuOH/H₂O (2/1/1, v/v/v, 2 mL) solution and 10% Pd/C (50 mg) was added to the solution. After being stirred in hydrogen (4 atm) for 36 h, the mixture was filtered, washed with water, and concentrated. A crude product was purified using a Sep-Pak column C18 (Macherey-Nagel, Düren, Germany) with water and methanol as eluents to obtain compound 5 (11.9 mg, 23.03 μmol, two-step yield: 60%). ¹H NMR (600 MHZ, Deuterium Oxide) δ 4.84 (d, J=4.2 Hz, 1H, 1-H), 4.65 (d, J=8.5 Hz, 1H, 1′-H), 4.31 (d, J=4.7 Hz, 1H, 4-H), 4.13 (q, J=6.6 Hz, 1H, 5-H), 3.98 (dd, J=10.6, 4.7 Hz, 1H, 3-H), 3.80 (dd, J=10.6, 4.0 Hz, 1H, 2-H), 3.63 (q, J=7.8, 6.9 Hz, 2H, 2′, Linker-OCH₂), 3.57-3.45 (m, 4H, 3′-H, 4′-H, 5′-H, Linker-OCH₂), 2.96 (t, J=7.7 Hz, 2H, Linker-NCH₂), 2.03 (s, 3H, NHAc-CH₃), 2.00-1.96 (m, 6H, NHAc-CH₃), 1.64 (tq, J=14.1, 6.9, 6.0 Hz, 4H, Linker-CH₂), 1.41 (dh, J=14.1, 6.8 Hz, 2H, Linker-CH₂), 1.15 (d, J=5.6 Hz, 3H, 6′-H), 1.04 (d, J=6.5 Hz, 3H, 6-H). ¹³C NMR (151 MHz, Deuterium Oxide) δ 174.8 (NH—C═O), 174.6 (NH—C═O), 101.0 (1′-C), 98.2 (1-C), 77.0 (3-C), 71.5-71.1 (3′-C, 5′-C), 68.1 (Linker-OCH₂), 67.1 (2-C), 65.5 (5-C), 57.1 (4′-H), 56.5 (2′-H), 52.5 (4-H), 39.4 (Linker-NCH₂), 28.1 (Linker-CH₂), 26.5 (Linker-CH₂), 22.3 (NHAc-CH₃), 22.2 (Linker-CH₂), 22.1 (NHAc-CH₃), 21.9 (NHAc-CH₃), 16.9 (6′-CH₃), 15.4 (6-CH₃). HR-ESI-MS (m/z): calcd for C₂₃H₄₃O₉N₄⁺ (M+H)⁺: 519.3025 found: 519.3052.

Compound 8: Compound 16 (30 mg, 51.0 μmol) was dissolved in a mixed solution of THF/AC₂O/AcOH (3/2/1, v/v/v, 3 mL) and freshly activated Zn (1 g) was added. After being stirred overnight at room temperature, the mixture was diluted with DCM and filtered. A filtrate was washed with a saturated NaHCO₃ aqueous solution and saturated brine. A combined organic layer was dried over anhydrous Na₂SO₄, filtered, evaporated under vacuum, and purified by silica gel column chromatography (DCM/MeOH=50/1 to 10/1, v/v) to obtain a compound intermediate. Then the compound intermediate was dissolved in a DCM/t-BuOH/H₂O (2/1/1, v/v/v, 2 mL) solution and 10% Pd/C (50 mg) was added to the solution. After being stirred in hydrogen (4 atm) for 36 h, the mixture was filtered, washed with water, and concentrated. A crude product was purified using a Sep-Pak column C18 (Macherey-Nagel, Düren, Germany) with water and methanol as eluents to obtain compound 8 (7.5 mg, 26.0 μmol, two-step yield: 51%). ¹H NMR (600 MHZ, Deuterium Oxide) δ 4.87-4.83 (m, 1H, 1-H), 4.19 (d, J=4.6 Hz, 1H, 4-H), 4.16 (q, J=6.6 Hz, 1H, 5-H), 3.95 (ddd, J=10.6, 4.9, 1.8 Hz, 1H, 3-H), 3.67-3.60 (m, 2H, 2-H, Linker-OCH₂), 3.46 (m, J=11.4, 7.7, 3.7 Hz, 1H, Linker-OCH₂), 2.95 (t, J=7.7 Hz, 2H, Linker-NCH₂), 2.04 (s, 3H, NHAc-CH₃), 1.62 (dp, J=20.4, 6.9 Hz, 4H, Linker-CH₂), 1.48-1.33 (m, 2H, Linker-CH₂), 1.05 (dd, J=6.6, 1.9 Hz, 3H, 6-CH₃). ¹³C NMR (151 MHZ, Deuterium Oxide) δ 175.6 (NH—C═O), 98.3 (1-H), 68.7 (2-H), 68.4 (3-H), 68.1 (Linker-OCH₂), 65.3 (5-H), 54.0 (4-H), 39.4 (Linker-NCH₂), 28.1 (Linker-CH₂), 26.5 (Linker-CH₂), 22.4 (Linker-CH₂), 21.9 (NHAc-CH₃), 15.6 (6-CH₃). HR-ESI-MS (m/z): calcd for C₁₃H₂₇O₅N₂+ (M+H)⁺: 291.1914 found: 291.1952.

Compound 20: Under nitrogen protection, compound 15 (180 mg, 0.25 mmol) was dissolved in a solution of pyridine (8 mL). Subsequently, water (2 mL), Et₃N (1.51 ml, 10.87 mmol), and 1,3-propanedithiol (1.48 ml, 14.8 mmol) were added to the reaction system, and stirred at room temperature for 6 h. The reaction mixture was concentrated and a residue was purified by silica gel column chromatography (DCM:MeOH=20:1, v/v) to obtain an amino sugar. Subsequently, sodium bicarbonate (62 mg, 0.74 mmol) was added to a solution of (R)-3-O-benzyl butyric acid 18 (96 mg, 0.49 mmol) and the amino sugar in acetonitrile (20 mL), and stirred at room temperature. After 10 min, HOBt (6.7 mg, 49.4 μmol) and EDC.HCl (57 mg, 0.30 mmol) were sequentially added and stirred at the same temperature for 6 h. After TLC detected that the reaction was completed, a crude product was dissolved in ethyl acetate and washed with a saturated sodium chloride solution. A separated organic layer was dried over anhydrous Na₂SO₄, concentrated, and purified by column chromatography (petroleum ether: ethyl acetate=1:1) to obtain compound 20 (188.9 mg, 0.22 mmol, 87% yield). [α]²⁵_{D}=+114.5° (c=0.3, CHCl₃); IR vmax (film) 3029, 2938, 2109, 1697, 1539, 1454, 1361, 1217, 1102, 1044, 819, 756, 698 cm⁻¹; ¹H NMR (400 MHZ, Chloroform-d) δ 7.88-7.73 (m, 4H, Ar—H), 7.55-7.14 (m, 24H, Ar—H), 6.73 (d, J=10.3 Hz, 1H, NHAc-H), 5.20 (d, J=7.9 Hz, 2H, Ar—CH₂), 4.99 (d, J=11.3 Hz, 1H, Ar—CH₂), 4.66 (td, J=13.8, 12.6, 3.0 Hz, 3H, Ar—CH₂,4-H), 4.59-4.42 (m, 6H, Ar—CH₂, 1-H), 4.09-3.93 (m, 3H, 5-H, 3-H, RHb-3), 3.63-3.47 (m, 1H, Linker-OCH₂), 3.42-3.14 (m, 4H, Linker-OCH₂, Linker-NCH₂, 2-H), 2.66-2.47 (m, 2H, RHb-2), 1.65-1.50 (m, 4H, Linker-CH₂), 1.27 (d, J=6.3 Hz, 5H, Linker-CH₂, RHb-4), 1.13 (d, J=6.4 Hz, 3H, 6-CH₃). ¹³C NMR (101 MHZ, Chloroform-d) δ 171.6 (NH—C═O), 138.7 (Ar), 138.1 (Ar), 137.9 (Ar), 136.8 (Ar), 136.1 (Ar), 133.3 (Ar), 132.9 (Ar), 128.5 (Ar), 128.5 (Ar), 128.4 (Ar), 128.3 (Ar), 128.0 (Ar), 127.9 (Ar), 127.8 (Ar), 127.8 (Ar), 127.8 (Ar), 127.7 (Ar), 127.6 (Ar), 127.6 (Ar), 127.5 (Ar), 127.3 (Ar), 127.2 (Ar), 126.6 (Ar), 126.3 (Ar), 125.8 (Ar), 125.6 (Ar), 97.4 (1-H), 75.9 (2-C), 73.0 (Ar—CH₂), 72.7, 71.5 (Ar—CH₂), 70.7 (Ar—CH₂), 68.2 (Linker-OCH₂), 67.2 (Ar—CH₂), 64.5 (5-H), 50.4 (Ar—CH₂,4-H), 47.2 (Linker-NCH₂), 43.7 (RHb-2), 29.1 (Linker-CH₂), 27.6 (Linker-CH₂), 23.5 (Linker-CH₂), 18.9 (RHb-4), 16.8 (6-CH₃). HR-ESI-MS (m/z): calcd for C₅₅H₆₃O₈N₂⁺ (M+H)⁺: 879.4579 found: 879.4635

Compound 21: Under nitrogen protection, compound 15 (150 mg, 0.21 mmol) was dissolved in a solution of pyridine (8 mL). Subsequently, water (2 mL), Et₃N (1.26 ml, 9.06 mmol), and 1,3-propanedithiol (1.24 mL, 12.36 mmol) were added to the reaction system, and stirred at room temperature for 6 h. The reaction mixture was concentrated and a residue was purified by silica gel column chromatography (DCM:MeOH=20:1, v/v) to obtain an amino sugar. Subsequently, sodium bicarbonate (52 mg, 0.62 mmol) was added to a solution of(S)-3-O-benzyl butyric acid 19 (80 mg, 0.41 mmol) and the amino sugar in acetonitrile (17 mL), and stirred at room temperature. After 10 min, HOBt (5.6 mg, 41.2 μmol) and EDC (64 mg, 0.33 mmol) were added sequentially and stirred at the same temperature for 6 h. After TLC detected that the reaction was completed, a crude product was dissolved in ethyl acetate and washed with a saturated sodium chloride solution. A separated organic layer was dried over anhydrous Na₂SO₄, concentrated, and purified by column chromatography (petroleum ether: ethyl acetate=1:1) to obtain compound 21 (150.2 mg, 0.17 mmol, 83% yield). [α]²⁵_{D}=+80.1° (c=0.75, CHCl₃); IR vmax (film) 3029, 2942, 2868, 2109, 1697, 1532, 1454, 1361, 1217, 1104, 1045, 818, 755, 698 cm⁻¹; ¹H NMR (400 MHZ, Chloroform-d) δ 7.85-7.70 (m, 4H Ar—H), 7.54-7.13 (m, 24H Ar—H), 6.59 (d, J=10.1 Hz, 1H, NHAc—H), 5.17 (d, J=8.5 Hz, 2H, Ar—CH₂), 4.95 (d, J=11.3 Hz, 1H, Ar—CH₂), 4.61 (m, 4H, 4-H.1-H. Ar—CH₂), 4.43 (m, 5H, Ar—CH₂), 4.10-3.84 (m, 3H, 5-H, 3-H, RHb-3), 3.54 (d, J=9.4 Hz, 1H, Linker-OCH₂), 3.40-3.13 (m, 4H, Linker-OCH₂, Linker-NCH₂, 2-H), 2.54 (m, J=5.4, 2.1 Hz, 2H, RHb-2), 1.54 (s, 4H, Linker-CH₂), 1.37-1.19 (m, 5H, Linker-CH₂, RHb-4), 1.15 (d, J=6.4 Hz, 3H, 6-CH₃). ¹³C NMR (101 MHZ, Chloroform-d) δ 171.7 (NH—C═O), 138.7 (Ar), 138.2 (Ar), 137.9 (Ar), 136.1 (Ar), 133.3 (Ar), 132.9 (Ar), 128.5 (Ar), 128.4 (Ar), 128.3 (Ar), 128.2 (Ar), 128.0 (Ar), 127.9 (Ar), 127.88 (Ar), 127.82 (Ar), 127.6 (Ar), 127.5 (Ar), 127.45 (Ar), 127.3 (Ar), 127.2 (Ar), 126.6 (Ar), 126.3 (Ar), 125.9 (Ar), 125.7 (Ar), 97.3 (1-C), 76.0 (3-C) 75.98 (2-C), 72.8 (RHb-3), 72.5 (Ar—CH₂), 71.4 (Ar—CH₂), 70.4 (Ar—CH₂), 68.2 (Linker-OCH₂), 67.1 (Ar—CH₂), 64.5 (5-C), 50.2 (4-C, Ar—CH₂), 46.2 (Linker-NCH₂), 43.7 (RHb-2), 29.1 (Linker-CH₂), 23.5 (Linker-CH₂), 19.7 (RHb-4), 16.9 (6-CH₃). HR-ESI-MS (m/z): calcd for C₅₅H₆₂O₈N₂Na⁺ (M+Na)⁺: 901.4398 found: 901.4446.

Compound 9: Compound 20 (30 mg, 34.15 μmol) was dissolved in a mixed solution of DCM/t-BuOH/H₂O (2/1/1, v/v/v), and then 10% Pd/C (50 mg) was added. After being stirred for 36 h under a hydrogen (4 atm) atmosphere, the mixture was filtered and washed with water. Then, a residue was purified using a Sep-Pak column C18 (Macherey-Nagel, Germany) with water and methanol as eluents to obtain compound 9 (10.2 mg, 30.4 μmol, 89%). ¹H NMR (400 MHz, Deuterium Oxide) δ 4.79 (d, J=3.9 Hz, 1H, 1-H), 4.12 (m, J=20.1, 13.5, 5.7 Hz, 3H, 4-H, 5-H, RHb-3), 3.90 (dd, J=10.5, 4.3 Hz, 1H, 3-H), 3.58 (m, J=8.9, 5.8, 4.9 Hz, 2H, 2-H, Linker-OCH₂), 3.40 (dt, J=10.6, 6.2 Hz, 1H, Linker-OCH₂), 2.89 (t, J=7.6 Hz, 2H, Linker-NCH₂), 2.46-2.36 (m, 2H, RHb-2), 1.57 (dq, J=12.6, 6.7, 5.5 Hz, 4H, Linker-CH₂), 1.33 (dp, J=13.5, 6.6 Hz, 2H, Linker-CH₂), 1.13 (d, J=6.1 Hz, 3H, RHb-4), 1.00 (d, J=6.3 Hz, 3H, 6-CH₃). ¹³C NMR (101 MHZ, Deuterium Oxide) δ 98.3 (1-H), 68.6 (2-H), 68.4 (3-H), 68.0 (Linker-OCH₂), 65.0 (5-C, RHb-3), 53.8 (4-C), 44.7 (RHb-2), 39.3 (Linker-NCH₂), 28.0 (Linker-CH₂), 26.5 (Linker-CH₂), 22.4 (Linker-CH₂), 22.1 (RHb-4), 15.5 (6-CH₃). HR-ESI-MS (m/z): calcd for C₁₅H₃₁O₆N₂+ (M+H)⁺: 335.2177 found: 335.2219.

Compound 11: Compound 21 (30 mg, 34.15 μmol) was dissolved in a mixed solution of DCM/t-BuOH/H₂O (2/1/1, v/v/v, 2 mL), and 10% Pd/C (50 mg) was added. After being stirred in hydrogen (4 atm) for 36 h, the mixture was filtered and washed with water. A residue was purified using a Sep-Pak column C18 (Macherey-Nagel, Germany) with water and methanol as eluents to obtain compound 11 (10.4 mg, 31.08 μmol, 91% yield). ¹H NMR (400 MHZ, Deuterium Oxide) δ 4.92 (d, J=4.0 Hz, 1H, 1-H), 4.26 (m, J=19.2, 5.4 Hz, 3H, 4-H, 5-H, RHb-3), 4.02 (dd, J=10.5, 4.5 Hz, 1H, 3-H), 3.78-3.65 (m, 2H, 2-H, Linker-OCH₂), 3.53 (dt, J=9.9, 6.2 Hz, 1H, Linker-OCH₂), 3.02 (t, J=7.6 Hz, 2H, Linker-NCH₂), 2.61-2.48 (m, 2H, RHb-2), 1.76-1.65 (m, 4H, Linker-CH₂), 1.56-1.39 (m, 2H, Linker-CH₂), 1.25 (d, J=6.2 Hz, 3H, RHb-4), 1.13 (d, J=6.4 Hz, 3H, 6-CH₃). ¹³C NMR (101 MHz, Deuterium Oxide) δ 175.3 (NH—C═O), 98.3 (1-C), 68.6 (3-C), 68.4 (2-C), 68.0 (Linker-OCH₂), 65.2-65.0 (5-C, RHb-3), 53.9 (4-C), 44.5 (RHb-2), 39.3 (Linker-NCH₂), 28.0 (Linker-CH₂), 26.5 (Linker-CH₂), 22.4 (Linker-CH₂), 21.9 (RHb-4), 15.6 (6-CH₃). HR-ESI-MS (m/z): calcd for C₁₅H₃₁O₆N₂⁺ (M+H)⁺: 335.2177 found: 335.2214.

Compound 22: H₂O (1 mL) was added to a solution of compound 20 (150 mg, 0.17 mmol) in DCM (2.0 mL), followed by addition of DDQ (58 mg, 0.26 mmol). The reaction mixture was stirred at room temperature for 5 h, after which TLC indicated that the reaction was complete. The mixture was diluted with DCM (2×10 mL) and washed with saturated NaHCO₃ (20 mL). An organic layer was dried over anhydrous Na₂SO₄, and the reaction mixture was concentrated under vacuum. A residue was purified by silica gel column chromatography (petroleum ether: acetone=1:1) to obtain compound 22 (116.7 mg, 0.16 mmol, 93% yield). [α]²⁵_{D}=+33° (c=1.2, CHCl₃); IR vmax (film) 3030, 2923, 1697, 1540, 1453, 1361, 1216, 1100, 1037, 819,736, 697 cm⁻¹; ¹H NMR (600 MHZ, Chloroform-d) δ 7.39-7.13 (m, 20H, Ar—H), 6.72 (d, J=9.4 Hz, 1H, NH—H), 5.17 (d, J=16.9 Hz, 2H, Ar—CH₂), 4.59 (d, J=11.3 Hz, 1H, Ar—CH₂), 4.55-4.42 (m, 6H, Ar—CH₂, 1-H), 4.30 (d, J=8.1 Hz, 1H, 4-H), 4.14-4.07 (m, 1H, 3-H), 4.03 (d, J=12.2 Hz, 1H, 5-H), 3.97 (qd, J=6.4, 3.8 Hz, 1H, RHb-3), 3.52 (d, J=25.7 Hz, 1H, linker-OCH₂), 3.30-3.14 (m, 4H, 2-H, linker-OCH₂, linker-NCH₂), 2.61-2.44 (m, 2H, RHb-2), 1.59-1.47 (m, 4H, linker-CH₂), 1.36-1.25 (m, 5H, RHb-4, linker-CH₂), 1.06 (d, J=6.4 Hz, 3H, 6-CH₃). ¹³C NMR (151 MHz, Chloroform-d) δ 172.9 (NH—C═O), 156.8 (Cbz-C═O), 156.2 (Cbz-C═O), 138.3 (Ar), 138.0 (Ar), 137.9 (Ar), 128.6 (Ar), 128.5 (Ar), 128.45 (Ar), 128.4 (Ar), 128.37 (Ar), 127.9 (Ar), 127.8 (Ar), 127.77 (Ar), 127.75 (Ar), 127.7 (Ar), 127.72 (Ar), 127.3 (Ar), 127.2 (Ar), 97.0 (1-C), 77.1 (2-C) 72.5 (RHb-3), 72.4 (Ar—CH₂), 70.7 (Ar—CH₂), 69.8 (3-C), 68.2 (linker-OCH₂), 67.2 (linker-OCH₂), 64.2 (5-C), 53.7 (4-C), 50.3 (Ar—CH₂), 47.2 (linker-NCH₂), 46.2 (linker-NCH₂), 43.5 (RHb-2-CH₂), 29.2 (linker-CH₂), 28.0 (linker-CH₂), 27.5 (linker-CH₂), 23.5 (linker-CH₂), 19.0 (RHb-4), 16.7 (6-CH₃). HR-ESI-MS (m/z): calcd for C₄₄H₅₄O₈N₂Na⁺ (M+Na)⁺: 761.3772 found: 761.3807.

Compound 23: Selenoglycoside 12 (156 mg, 0.26 mmol) and acceptor 22 (94.3 mg, 0.13 mmol) were mixed, azeotropically evaporated with toluene (3×5 mL) to remove water, and dried under vacuum using an oil pump for 2 h. The donor-acceptor mixture was dissolved in DCM (8 mL) at 0° C., a freshly activated 4 Å molecular sieve was added, and then NIS (57.4 mg, 0.26 mmol) and TfOH (22.6 μL, 0.26 mmol) were slowly added. After stirring for 4 h, a drop of triethylamine (Et₃N) was added to the reaction system for neutralization, and the 4 Å molecular sieve was filtered. A filtrate was washed with a 10% Na₂S₂O₃ solution and a saturated NaHCO₃ solution, respectively. A combined organic layer was dried over anhydrous Na₂SO₄, filtered, and concentrated. A crude product was purified by silica gel column chromatography (petroleum ether:ethyl acetate=2:1) to obtain compound 23 (109.7 mg, 0.09 mmol, 72% yield). [α] 25)=+60.9° (c=1.1, CHCl₃); IR vmax (film) 3029, 2938, 2870, 2107, 1681, 1525, 1454, 1361, 1217, 1093, 1042, 819, 755, 698 cm⁻¹; ¹H NMR (400 MHZ, Chloroform-d) δ 7.85-7.73 (m, 4H), 7.51-7.12 (m, 27H, Ar), 6.73 (d, J=10.1 Hz, 1H, NHAc), 6.64 (d, J=7.7 Hz, 1H, NHAc), 5.17 (m, 2H, Ar-CH₂), 4.96 (d, J=7.5 Hz, 1H, 1′-H), 4.90 (d, J=10.8 Hz, 1H, Ar—CH₂), 4.78 (d, J=10.9 Hz, 1H, Ar—CH₂), 4.60 (d, J=11.1 Hz, 1H, Ar—CH₂), 4.47 (m, 2H, Ar—CH₂), 4.43 (m, 2H, 1-H,4-H), 4.35 (d, J=11.9 Hz, 1H, Ar—CH₂), 4.24 (d, J=12.0 Hz, 1H, Ar—CH₂), 4.09 (dd, J=10.2, 4.6 Hz, 1H, 2-H), 3.97 (td, J=6.6, 3.6 Hz, 2H, RHb-3, 5-H), 3.72 (m, 2H, 2′-H, 4′-H), 3.49 (s, 1H, Linker-OCH₂), 3.26 (m, 3H, Linker-OCH₂, 3′-H, 5′-H), 3.17 (m, 3H, 3-H, Linker-NCH₂), 2.57 (dd, J=15.3, 3.6 Hz, 1H, RHb-2), 2.47 (dd, J=15.3, 7.0 Hz, 1H, RHb-2), 1.52 (s, 4H, Linker-CH₂), 1.35 (dd, J=9.9, 5.9 Hz, 6H, 6′-CH₃, RHb-4), 1.27 (s, 2H, Linker-CH₂), 1.06 (d, J=6.4 Hz, 3H, 6-CH₃). ¹³C NMR (101 MHZ, Chloroform-d) δ 171.5 (NH—C═O), 161.4 (NH—C═O), 138.4 (Ar), 138.1 (Ar), 137.9 (Ar), 136.7 (Ar), 134.8 (Ar), 133.3 (Ar), 133.1 (Ar), 128.6 (Ar), 128.5 (Ar), 128.5 (Ar), 128.4 (Ar), 128.2 (Ar), 128.0 (Ar), 127.97 (Ar), 127.9 (Ar), 127.8 (Ar), 127.7 (Ar), 127.66 (Ar), 127.3 (Ar), 127.2 (Ar), 127.0 (Ar), 126.1 (Ar), 126.0 (Ar), 99.3 (1′-H), 96.4 (1-H), 92.6, 79.8 (4′-C), 77.2 (3-C), 74.4 (Ar—CH₂), 73.7 (2-C), 72.9 (5-C), 72.1 (Ar—CH₂), 70.8 (5′-C), 70.7 (Ar—CH₂), 68.1 (Ar—CH₂), 68.0 (3′-C), 67.2 (Ar—CH₂), 65.0 (RHb-3), 58.4 (2′-H), 52.7 (4-H), 50.3 (Ar—CH₂), 47.3 (Linker-NCH₂), 43.3 (RHb-2), 29.1 (Linker-CH₂), 27.6 (Linker-CH₂), 23.4 (Linker-CH₂), 19.3 (RHb-4), 18.5 (6′-CH₃), 16.5 (6-CH₃). HR-ESI-MS (m/z): calcd for C₆₃H₇₁O₁₁N₆Cl₃Na⁺ (M+Na)⁺: 1215.4139 found: 1215.4149.

Compound 6: Compound 23 (50 mg, 41.9 μmol) was dissolved in a mixed solution of THF/AC₂O/AcOH (3/2/1, v/v/v, 3 mL) and freshly activated Zn (1 g) was added. After being stirred overnight at room temperature, the mixture was diluted with DCM and filtered. A filtrate was washed with a saturated NaHCO₃ aqueous solution and saturated brine. A combined organic layer was dried over anhydrous Na₂SO₄, filtered, evaporated under vacuum, and purified by silica gel column chromatography (DCM/MeOH=50/1 to 10/1, v/v) to obtain a compound intermediate. Then the compound intermediate was dissolved in a DCM/t-BuOH/H₂O (2/1/1, v/v/v, 2 mL) solution and 10% Pd/C (50 mg) was added to the solution. After being stirred in hydrogen (4 atm) for 36 h, the mixture was filtered, washed with water, and concentrated. A crude product was purified using a Sep-Pak column C18 (Macherey-Nagel, Düren, Germany) with water and methanol as eluents to obtain compound 6 (12 mg, 21.4 μmol, two-step yield: 51% yield). ¹H NMR (400 MHZ, Deuterium Oxide) δ 4.70 (d, 1H, 1-H), 4.60 (d, J=8.2 Hz, 1H, 1′-H), 4.27 (d, J=4.7 Hz, 1H, 4-H), 4.08 (p, J=6.5, 6.0 Hz, 2H, 5-H, RHb-3), 3.93 (dd, J=10.4, 4.7 Hz, 1H, 3-H), 3.72 (dd, J=10.4, 3.9 Hz, 1H, 2-H), 3.57 (dt, J=9.6, 6.5 Hz, 2H, 2′-H, Linker-OCH₂), 3.53-3.48 (m, 2H, 3′-H, Linker-OCH₂, 4′-H, 5′-H), 2.90 (t, J=7.6 Hz, 2H, Linker-NCH₂), 2.38 (h, J=8.7, 8.1 Hz, 2H, RHb-2), 1.82 (m, 6H, NHAc-CH₃), 1.58 (m, J=9.1, 8.5 Hz, 4H, Linker-CH₂), 1.36 (m, J=7.5, 7.1 Hz, 2H, Linker-CH₂), 1.15 (d, J=6.1 Hz, 3H, RHb-4), 1.08 (d, J=4.5 Hz, 3H, 6′-CH₃), 0.98 (d, J=6.4 Hz, 3H, 6-CH₃). ¹³C NMR (101 MHz, Deuterium Oxide) δ 174.6 (NH—C═O), 101.2 (1′-C), 98.1 (1-C), 76.8 (5-C), 71.7-70.9 (3′-C, 5′-C), 68.0 (Linker-OCH₂), 67.6 (2-C), 65.3-65.1 (5-C, RHb-3), 57.0 (4′-C), 56.4 (2′-C), 52.7 (4-C), 44.8 (RHb-2), 39.3 (Linker-NCH₂), 28.0 (Linker-CH₂), 26.4 (Linker-CH₂), 22.3 (Linker-CH₂), 22.1 (NHAc), 22.1 (NHAc), 22.0 (RHb-4), 16.9 (6′-C), 15.4 (6-C). HR-ESI-MS (m/z): calcd for C₂₅H₄₇O₁₀N₄⁺(M+H)⁺: 563.3287 found: 563.3349.

### Example 3

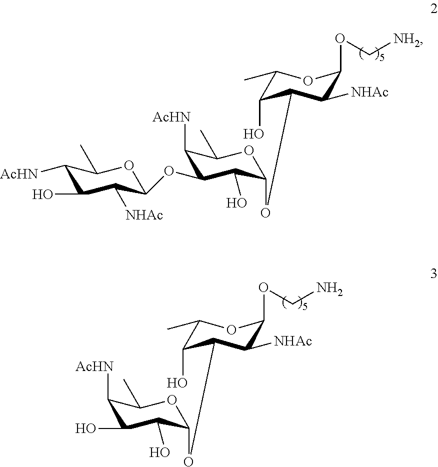

Compounds 2 and 3 were synthesized, as shown in FIG. 5.

Compound 24 was synthesized by a known method (Cai Juntao, doctoral thesis, Jiangnan University, 2020). 2-Naphthalene methylene was selectively removed from compound 24 by DDQ to obtain disaccharide acceptor 25. The donor selenoglycoside 12 and acceptor 25 were catalyzed by TMSOTf and NIS to obtain trisaccharide 26. Subsequently, zinc powder, acetic acid, and acetic anhydride were used for conducting reductive acylation and catalytic hydrogenation on compounds 26 and 25, respectively to obtain deprotected target compounds 2 and 3.

### Specific Experimental Operations and Steps:

Compound 25: H₂O (2 mL) was added to a solution of compound 24 (380 mg, 0.38 mmol) in DCM (10 mL), followed by addition of DDQ (126 mg, 0.57 mmol). The reaction mixture was stirred at room temperature for 5 h, after which TLC indicated that the reaction was complete. The mixture was diluted with DCM (2×10 mL) and washed with saturated NaHCO₃ (20 mL). An organic layer was dried over anhydrous Na₂SO₄, and the reaction mixture was concentrated under vacuum. A residue was purified by silica gel column chromatography (petroleum ether:ethyl acetate=1:1) to obtain compound 25 (213 mg, 0.25 mmol, 65% yield). [α]²⁵_{D}=−16.2° (c=0.8, CHCl₃); IR vmax (film) 3029, 2939, 2876, 2108, 1697, 1540, 1454, 1361, 1228, 1089, 1045, 830, 755, 699 cm⁻¹; ¹H NMR (600 MHZ, Chloroform-d) δ 7.46-7.09 (m, 24H, Ar), 6.36 (s, 1H, NHAc-H), 5.18-5.13 (m, 2H, Ar—CH₂), 5.04 (d, J=3.5 Hz, 1H, 1′-H), 4.96 (s, 1H, 1-H), 4.84 (d, J=11.8 Hz, 1H, Ar—CH₂), 4.70 (s, 2H, Ar—CH₂), 4.64 (d, J=11.7 Hz, 1H, Ar—CH₂), 4.47 (d, J=9.3 Hz, 2H, Ar—CH₂), 4.39 (s, 1H, 2′-H), 4.19-4.11 (m, 2H, 3-H, 5-H), 3.82 (m, 2H, 3′-H, 5′-H), 3.77 (dd, J=9.8, 3.3 Hz, 1H, 2-H), 3.71 (dd, J=3.8, 1.5 Hz, 1H, 4-H), 3.57 (m, 2H, 4′-H, Linker-OCH₂), 3.38 (m, 1H, Linker-OCH₂), 3.19 (m, 2H, Linker-NCH₂), 1.62 (s, 3H, NHAc-CH₃), 1.55 (s, 4H, Linker-CH₂), 1.34-1.24 (m, 2H, Linker-CH₂), 1.15 (d, J=6.4 Hz, 3H, 6-H), 1.13-1.08 (m, 3H, 6′-H). ¹³C NMR (101 MHZ, Chloroform-d) δ 170.5 (NH—C═O), 138.5 (Ar), 137.8 (Ar), 137.0 (Ar), 128.9 (Ar), 128.8 (Ar), 128.76 (Ar), 128.6 (Ar), 128.5 (Ar), 128.3 (Ar), 128.0 (Ar), 127.8 (Ar), 127.7 (Ar), 127.6 (Ar), 127.4 (Ar), 127.2 (Ar), 98.0 (1-H), 97.1 (1′-H), 78.5, 74.5 (Ar—CH₂), 70.1, 68.0 (Ar—CH₂), 67.2 (Ar—CH₂), 66.6, 66.4 (4-C), 66.3, 49.7 (2′-C), 47.1 (Linker-NCH₂), 29.3 (Linker-CH₂), 23.5 (Linker-CH₂), 22.8 (NHAc-CH₃), 17.2 (6-CH₃), 16.8 (6′-CH₃). HR-ESI-MS (m/z): calcd for C₄₈H₅₉O₁₀N₅Na⁺ (M+Na)⁺: 888.4154 found: 888.4215.

Compound 26: Selenoglycoside 12 (122.4 mg, 0.2 mmol) and acceptor 25 (86.5 mg, 0.1 mmol) were mixed, azeotropically evaporated with toluene (3×5 mL) to remove water, and dried under vacuum using an oil pump for 2 h. The donor-acceptor mixture was dissolved in DCM (8 mL) at 0° C., a freshly activated 4 Å molecular sieve was added, and then NIS (45 mg, 0.2 mmol) and TfOH (17.7 L, 0.2 mmol) were slowly added. After stirring for 4 h, a drop of triethylamine (Et₃N) was added to the reaction system for neutralization, and the 4 Å molecular sieve was filtered. A filtrate was washed with a 10% Na₂S₂O₃ solution and a saturated NaHCO₃ solution, respectively. A combined organic layer was dried over anhydrous Na₂SO₄, filtered, and concentrated. A crude product was purified by silica gel column chromatography (petroleum ether:ethyl acetate=2:1) to obtain compound 26 (95 mg, 0.072 mmol, 72%). [α]²⁵_{D}=−7.8° (c=0.9, CHCl₃); IR vmax (film) 3029, 2938, 2880, 2108, 1697, 1658, 1525, 1454, 1361, 1231, 1096, 1048, 822, 757, 699 cm⁻¹; ¹H NMR (400 MHZ, Methanol-d₄) δ 7.85-7.78 (m, 4H, Ar), 7.51-7.42 (m, 7H, Ar), 7.40-7.17 (m, 16H, Ar), 5.14 (d, J=12.3 Hz, 2H, Ar—CH₂), 4.98 (d, J=13.4 Hz, 3H, Ar—CH₂, 1″-H), 4.94-4.82 (m, 4H, Ar—CH₂, 1′-H, 1-H), 4.75 (dd, J=29.5, 11.7 Hz, 2H, Ar—CH₂), 4.64 (d, J=12.0 Hz, 1H, Ar—CH₂), 4.43 (s, 1H, 2-H), 4.24 (dd, J=10.1, 3.7 Hz, 1H, 3′-H), 4.14-4.07 (m, 1H, 5′-H), 4.04-3.83 (m, 5H, 3-H, 5-H, 2″-H, 3″-H, 4′-H), 3.76 (dd, J=10.0, 3.6 Hz, 1H, 2′-H), 3.69-3.49 (m, 2H, 4-H, Linker-OCH₂), 3.36 (m, 1H, Linker-OCH₂), 3.27 (dt, J=11.3, 5.9 Hz, 4H, Linker-OCH₂, 4″-H, 5″-H), 1.83-1.72 (m, 3H, NHAc), 1.53 (s, 4H, Linker-CH₂), 1.41-1.32 (m, 2H, Linker-CH₂), 1.29-1.24 (m, 3H, 6″-H), 1.23-1.16 (m, 3H, 6-H), 1.12 (s, 3H, 6′-H). ¹³C NMR (101 MHZ, Methanol-d₄) δ 172.1 (NH—C═O), 162.6 (NH—C═O), 138.9 (Ar), 138.0 (Ar), 135.2 (Ar), 133.2 (Ar), 128.3 (Ar), 128.2 (Ar), 127.9 (Ar), 127.6 (Ar), 127.3 (Ar), 127.0 (Ar), 126.2-124.6 (Ar), 101.0 (1″-H), 98.6 (1′-H), 97.1 (1-H), 79.8, 78.9 (4-C), 78.0 (3′-C), 75.0 (Ar—CH₂, 2′-C), 74.4, 70.5, 68.1, 67.0, 66.7 (Ar—CH₂), 65.3 (5′-H), 58.3, 49.0 (2-C), 47.8 (Linker-NCH₂), 28.5 (Linker-CH₂), 21.9 (NHAc-CH₃), 17.3 (6″-CH₃), 16.1 (6′-CH₃), 15.97 (6-CH₃). HR-ESI-MS (m/z): calcd for C₆₇H₇₆O₁₃N₉Cl₃Na⁺ (M+Na)⁺: 1342.4520 found: 1342.4535.

Compound 2: Compound 26 (30 mg, 22.7 μmol) was dissolved in a mixed solution of THF/AC₂O/AcOH (3/2/1, v/v/v, 3 mL) and freshly activated Zn (0.5 g) was added. After being stirred overnight at room temperature, the mixture was diluted with DCM and filtered. A filtrate was washed with a saturated NaHCO₃ aqueous solution and saturated brine. A combined organic layer was dried over anhydrous Na₂SO₄, filtered, evaporated under vacuum, and purified by silica gel column chromatography (DCM/MeOH=50/1 to 10/1, v/v) to obtain a compound intermediate. Then the compound intermediate was dissolved in a DCM/t-BuOH/H₂O (2/1/1, v/v/v, 2 mL) solution and 10% Pd/C (50 mg) was added to the solution. After being stirred in hydrogen (4 atm) for 36 h, the mixture was filtered, washed with water, and concentrated. A crude product was purified using a Sep-Pak column C18 (Macherey-Nagel, Düren, Germany) with water and methanol as eluents to obtain compound 2 (8 mg, 11.35 μmol, two-step yield: 50%). ¹H NMR (600 MHZ, Deuterium Oxide) δ 4.88 (d, J=4.4 Hz, 1H, 1′-H), 4.76 (d, 1-H), 4.55 (d, J=8.5 Hz, 1H, 1″-H), 4.24 (dd, J=4.8, 1.7 Hz, 1H, 4′-H), 4.17 (dd, J=11.1, 3.8 Hz, 1H, 2-H), 4.12 (tt, J=6.6, 3.7 Hz, 1H, 5′-H), 4.02-3.95 (m, 2H, 3′-H,5-H), 3.79 (dd, J=11.1, 3.2 Hz, 1H, 3-H), 3.70 (d, J=3.2 Hz, 1H, 4-H), 3.66-3.62 (m, 1H, 2′-H), 3.62-3.52 (m, 2H, 2″-H Linker-OCH₂), 3.48-3.32 (m, 4H, 3″-H, 4″-H, 5″-H, Linker-OCH₂), 2.87 (t, J=7.7 Hz, 2H, Linker-NCH₂), 1.94 (s, 3H, NHAc-CH₃), 1.90 (d, J=7.5 Hz, 9H, NHAc-CH₃), 1.60-1.47 (m, 4H, Linker-CH₂), 1.32 (tq, J=14.2, 7.5, 6.4 Hz, 2H, Linker-CH₂), 1.08 (dd, J=6.6 Hz, 3H, 6-H), 1.08 (dd, J=5.8 Hz, 3H, 6″-H), 0.94 (d, J=6.5 Hz, 3H, 6′-H). ¹³C NMR (151 MHZ, Deuterium Oxide) δ 174.8 (NH—C═O), 174.7 (NH—C═O), 101.6 (1″-H), 101.1 (1′-H), 97.1 (1-H), 76.8 (3-H,3′-H) 71.7 (4″-H), 71.4 (4-C), 71.0 (5″-H), 67.9 (Linker-OCH₂), 67.7 (2′-H), 66.5 (5-H), 66.1 (5′-C), 57.0 (4″- C), 56.3 (2″-C), 52.9 (4′-C), 48.5 (2-H), 39.4 (Linker-NCH₂), 28.0 (Linker-CH₂), 26.5 (Linker-CH₂), 22.3 (Linker-CH₂), 22.2 (NHAc-CH₃), 22.1 (NHAc-CH₃), 22.0 (NHAc-CH₃), 21.9 (NHAc-CH₃), 16.9 (6″-CH₃), 15.4 (6′-CH₃), 15.3 (6-CH₃). HR-ESI-MS (m/z): calcd for C₃₁H₅₆O₁₃N₅⁺ (M+H)⁺: 706.3869 found: 706.3881.

Compound 3: Compound 25 (50 mg, 57.8 μmol) was dissolved in a mixed solution of THF/AC₂O/AcOH (3/2/1, v/v/v, 3 mL) and freshly activated Zn (0.5 g) was added. After being stirred overnight at room temperature, the mixture was diluted with DCM and filtered. A filtrate was washed with a saturated NaHCO₃ aqueous solution and saturated brine. A combined organic layer was dried over anhydrous Na₂SO₄, filtered, evaporated under vacuum, and purified by silica gel column chromatography (DCM/MeOH=50/1 to 10/1, v/v) to obtain a compound intermediate. Then the compound intermediate was dissolved in a DCM/t-BuOH/H₂O (2/1/1, v/v/v, 2 mL) solution and 10% Pd/C (50 mg) was added to the solution. After being stirred in hydrogen (4 atm) for 36 h, the mixture was filtered, washed with water, and concentrated. A crude product was purified using a Sep-Pak column C18 (Macherey-Nagel, Düren, Germany) with water and methanol as eluents to obtain compound 3 (14.9 mg, 31.2 μmol, two-step yield: 54%). ¹H NMR (400 MHZ, Deuterium Oxide) δ 4.95 (d, J=4.1 Hz, 1H, 1′-H), 4.73 (d, J=3.4 Hz, 1H, 1-H), 4.25-4.14 (m, 3H, 2-H, 4′-H, 5′-H), 3.99 (dq, J=19.9, 5.2, 4.1 Hz, 2H, 5-H, 3′-H), 3.83 (dd, J=11.0, 3.1 Hz, 1H, 3-H), 3.74 (m, J=3.0 Hz, 1H, 4-H), 3.64-3.48 (m, 2H, Linker-OCH₂, 2′-H), 3.38 (dt, J=10.0, 6.2 Hz, 1H, Linker-OCH₂), 2.90 (t, J=7.6 Hz, 2H, Linker-NCH₂), 1.96 (d, J=30.7 Hz, 6H, NHAc), 1.59 (m, J=15.4, 7.7 Hz, 4H, Linker-CH₂), 1.34 (m, J=12.1, 7.5, 3.5 Hz, 2H, Linker-CH₂), 1.13 (d, J=6.6 Hz, 3H, 6-H), 1.00 (d, J=6.2 Hz, 3H, 6′-H). 13C NMR (101 MHz, Deuterium Oxide) δ 175.5 (NH—C═O), 174.6 (NH—C═O), 101.1 (1′-H), 97.0 (1-H), 76.5 (3-C), 71.3 (4-C), 68.5 (2′-C), 68.3 (3′-C), 67.8 (Linker-OCH₂), 66.4 (5-C), 65.8 (5′-C), 53.8 (4′-C) 48.4 (2-C), 39.3 (Linker-NCH₂), 28.0 (Linker-CH₂), 26.4 (Linker-CH₂), 22.2 (Linker-CH₂), 21.9 (NHAc-CH₃), 21.8 (NHAc-CH₃), 15.4 (6′-CH₃), 15.3 (6-CH₃). HR-ESI-MS (m/z): calcd for C₂₁H₄₀O₉N₃⁺ (M+H)⁺: 478.2759 found: 478.2819.

### Example 4

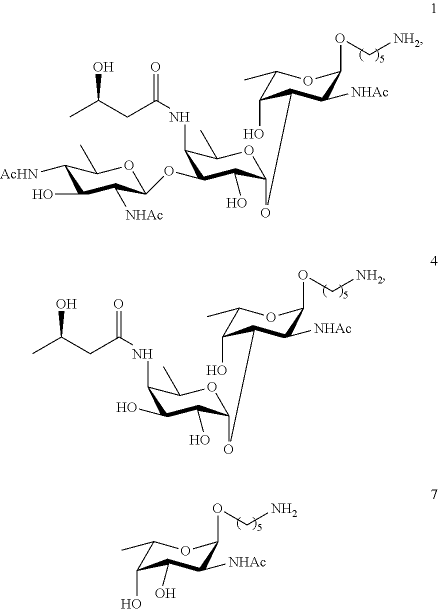

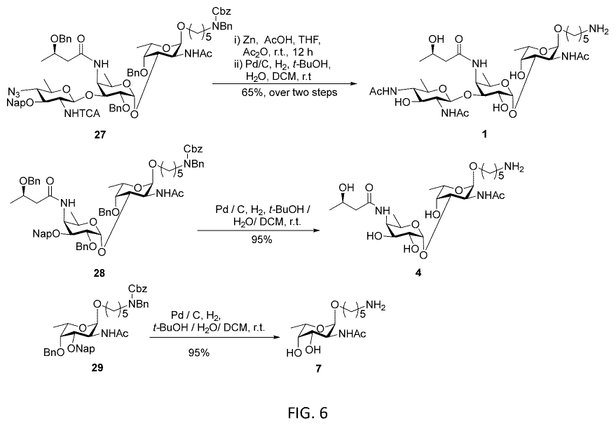

Compounds 1, 4, and 7 were synthesized, as shown in FIG. 6.

Compounds 27, 28, and 29 were synthesized by known methods (Cai Juntao, doctoral thesis, Jiangnan University, 2020), and catalytic hydrogenation was conducted on compounds 27, 28, and 29, respectively to obtain deprotected target compounds 1, 4, and 7.

### Specific Experimental Operations and Steps:

Compound 1: Trisaccharide 27 (30 mg, 20.41 μmol) was dissolved in a mixed solution of tetrahydrofuran, acetic anhydride, and acetic acid (3/2/1, v/v/v, 3 mL), newly activated Zn (1 g) was added, and the mixture was stirred overnight at room temperature. After TLC detected that the reaction of raw materials was complete, the reaction solution was diluted with dichloromethane and filtered. A filtrate was washed with a saturated sodium bicarbonate solution and a saturated sodium chloride solution. Subsequently, a combined organic layer was dried over anhydrous sodium sulfate, filtered, evaporated under vacuum, and dried under vacuum using an oil pump. A crude product was dissolved in a mixed solution of dichloromethane, tert-butyl alcohol, and water (3/6/1, v/v/v, 2 mL), and an appropriate amount of 10% palladium on carbon was added to the solution. The solution was stirred for 36 h under a hydrogen (4 atm) atmosphere. Subsequently, the mixture was filtered with diatomite and washed with water three times, and the solvent was evaporated in vacuum. A residue was purified by HPLC using a semi-preparative (Thermo Scientific Hypercarb) column at a flow rate of 1 mL/min, and was eluted with ultrapure water (solvent A) containing 0.1% formic acid and acetonitrile (solvent B) in a linear gradient of the solvent B (10% to 30%) for 30 min to obtain compound 1 (9.9 mg, 13.27 μmol, two-step yield: 65%). ¹H NMR (600 MHZ, Deuterium Oxide) δ 4.93 (t, J=3.3 Hz, 1H, 1′-H), 4.76 (d, J=3.3 Hz, 1H, 1-H), 4.60 (dd, J=7.8, 2.0 Hz, 1H, 1″-H), 4.33 (d, J=4.7 Hz, 1H, 4′-H), 4.23 (d, J=11.3, 3.1 Hz, 1H, 2-H), 4.18 (m, J=6.8 Hz, 1H, 5-H), 4.12 (q, J=6.1 Hz, 1H, RHb-3), 4.08-4.00 (m, 2H, 3′-H, 5′-H), 3.83 (dt, J=11.1, 2.9 Hz, 1H, 3-H), 3.75 (d, J=3.0 Hz, 1H, 4-H), 3.68 (dt, J=10.6, 3.2 Hz, 1H, 2′-H), 3.60 (m, J=7.9, 7.4 Hz, 2H, 2″-H, Linker-OCH₂), 3.44 (m, J=28.2, 9.7, 4.4 Hz, 4H, 3″-H, 4″-H, 5″-H, Linker-OCH₂), 2.98-2.89 (m, 2H, Linker-NCH₂), 2.45-2.36 (m, 2H, RHb-2), 1.94 (dt, J=6.4, 2.2 Hz, 9H, NHAc-CH₃), 1.66-1.54 (m, 4H, Linker-CH₂), 1.37 (q, J=7.4 Hz, 2H, Linker-CH₂), 1.18 (m, 3H, RHb-4), 1.15 (m, 3H, 6′-CH₃), 1.11 (m, 3H, 6″-CH₃) 1.00 (m, 3H, 6-CH₃). ¹³C NMR (151 MHz, Deuterium Oxide) δ 174.6 (NH—C═O), 171.0 (NH—C═O), 101.7 (1″-H), 101.1 (1′-H), 97.1 (1′-H), 76.7 (3′-C, 3-C), 71.8 (4-C), 71.4 (3″-C), 70.9 (5″-C), 67.8 (2′-H, Linker-OCH₂), 66.5 (5′-C), 66.0 (5-C), 65.2 (RHb-3) 57.0 (4″- C), 56.3 (2″-H), 44.8 (RHb-2), 39.3 (Linker-NCH₂), 28.0 (Linker-CH₂), 26.5 (Linker-CH₂), 22.3 (Linker-CH₂), 22.2 (NHAc-CH₃), 22.1 (NHAc-CH₃), 22.0 (NHAc-CH₃), 16.9 (6″-H), 15.5 (6-H), 15.3 (6′-CH₃). HR-ESI-MS (m/z): calcd for C₃₃H₅₉N₅O₁₄Na⁺ (M+Na)⁺: 772.3951 found: 772.3968.

Compound 4: Compound 28 (30 mg, 29.5 μmol) was dissolved in a mixed solution of DCM/t-BuOH/H₂O (2/1/1, v/v/v, 2 mL), and 10% Pd/C (50 mg) was added to the reaction system. After being stirred in hydrogen (4 atm) for 36 h, the mixture was filtered and washed with water. A residue was purified using a Sep-Pak column C18 (Macherey-Nagel, Germany) with water and methanol as eluents to obtain compound 4 (13.1 mg, 25.11 μmol, 85% yield). 1H NMR (600 MHz, Deuterium Oxide) δ 5.03 (d, J=4.0 Hz, 1H,1-H), 4.81 (s, 1H, 1′-H), 4.28 (d, J=12.9 Hz, 3H, 4-H, 5-H, 2′-H), 4.19 (q, J=6.3 Hz, 1H, RHb-3), 4.08 (dq, J=22.1, 5.2, 4.0 Hz, 2H, 3-H, 5′-H), 3.91 (dd, J=11.0, 3.0 Hz, 1H, 3′-H), 3.79 (d, J=3.2 Hz, 1H, 4′-H), 3.67 (dt, J=12.6, 6.7 Hz, 1H, linker-OCH₂), 3.63-3.58 (m, 1H, 2-H), 3.47 (dt, J=11.0, 6.3 Hz, 1H, linker-OCH₂), 2.99 (t, J=7.7 Hz, 2H, linker-NCH₂), 2.55-2.45 (m, 2H, RHb-2), 2.00 (s, 2H, NHAc), 1.65 (dq, J=23.2, 7.6 Hz, 4H, linker-CH₂), 1.42 (tq, J=14.6, 7.7, 7.1 Hz, 2H, linker-CH₂), 1.22 (dd, J=13.0, 6.2 Hz, 6H, RHb-4, 6′—H), 1.09 (s, 3H, 6-H). ¹³C NMR (151 MHz, Deuterium Oxide) δ 101.1 (1-C), 97.0 (1′-C), 76.5 (3′-C), 71.3 (4′-C), 68.6 (2-C), 68.4 (3-C), 67.8 (linker-OCH₂), 66.5 (5′-C), 65.8, 65.0 (RHb-3), 53.8, 48.5, 44.7 (RHb-2), 39.4 (linker-CH₂), 28.0 (linker-CH₂), 26.4 (linker-CH₂), 22.1-22.0 (NHAc, linker-CH₂, RHb-4), 15.5 (6-H), 15.3 (6′-H). HR-ESI-MS (m/z): calcd for C₂₃H₄₄O₁₀N₃⁺ (M+H)⁺: 522.3021 found: 522.3083.

Compound 7: Compound 29 (20 mg, 33.10 μmol) was dissolved in a mixed solution of DCM/t-BuOH/H₂O (2/1/1, v/v/v, 2 mL), and 10% Pd/C (50 mg) was added to the reaction system. After being stirred in hydrogen (4 atm) for 36 h, the mixture was filtered and washed with water. A residue was purified using a Sep-Pak column C18 (Macherey-Nagel, Germany) with water and methanol as eluents to obtain compound 7 (9.1 mg, 31.5 μmol, 95% yield). 1H NMR (400 MHZ, Deuterium Oxide) δ 4.85 (d, J=3.3 Hz, 1H, 1-H), 4.09 (q, J=7.9 Hz, 2H, 2-H, 5-H), 3.91 (d, J=11.1 Hz, 1H, 3-H), 3.81 (s, 1H, 4-H), 3.73-3.59 (m, 1H, linker-OCH₂), 3.47 (dt, J=11.1, 6.6 Hz, 1H, linker-OCH₂), 3.00 (t, J=7.8 Hz, 2H, linker-NCH₂), 2.04 (s, 3H, NHAc), 1.66 (dp, J=21.8, 8.0 Hz, 4H, linker-CH₂), 1.44 (p, J=7.9 Hz, 2H, linker-CH₂), 1.23 (d, J=6.6 Hz, 3H, 6-H). ¹³C NMR (101 MHz, Deuterium Oxide) δ 174.6 (NHAc-C═O), 96.9 (1-C), 71.1 (4-C), 67.8 (3-C, linker-OCH₂), 66.6 (5-C), 49.8 (2-C), 39.4 (linker-NCH₂), 28.0 (linker-CH₂), 26.5 (linker-CH₂), 22.3 (linker-CH₂), 22.0 (NHAc-CH₃), 15.5 (6-CH₃). HR-ESI-MS (m/z): calcd for C₁₃H₂₇O₅N₂⁺ (M+H)⁺: 291.1914 found: 291.1946.

### Example 5

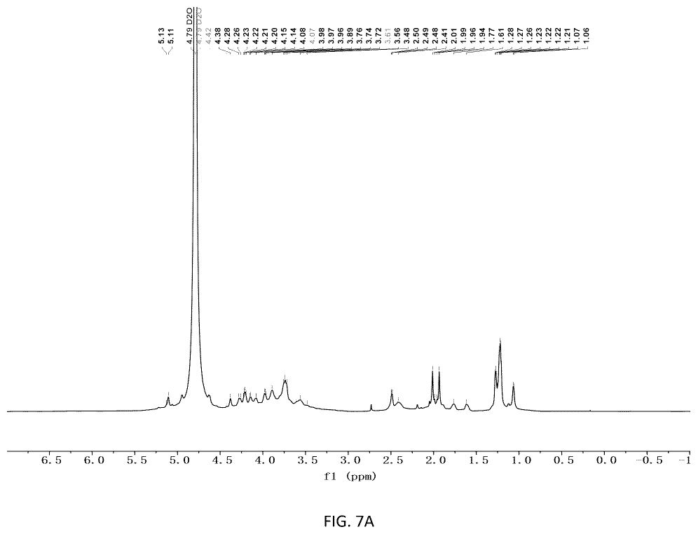

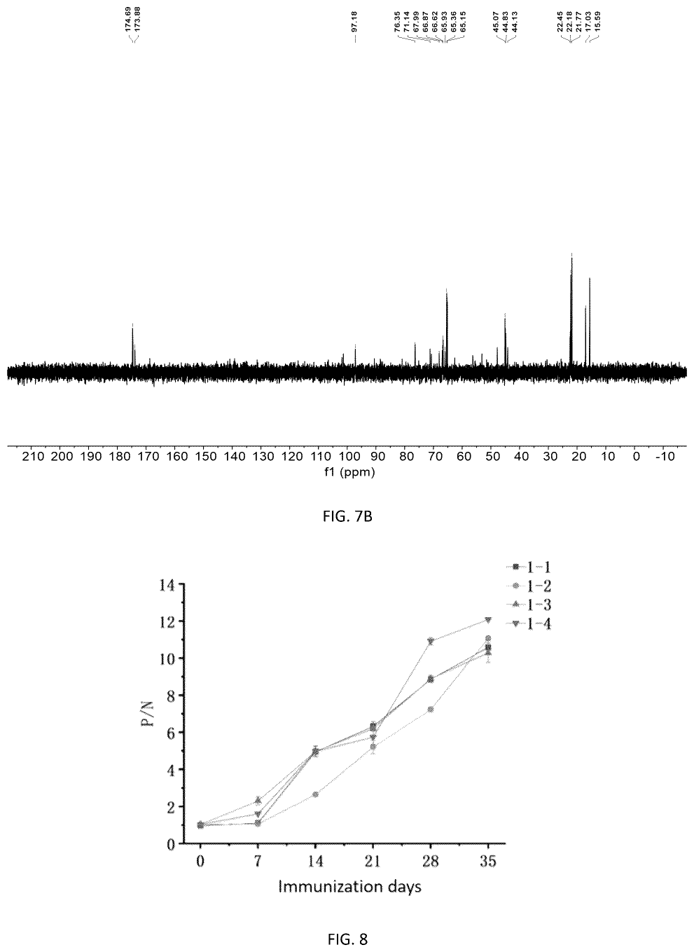

Lipopolysaccharide (LPS) and O-antigen (OPS) of V. cholerae O₁₀₀ serotype were extracted. The 1H-NMR and 13C-NMR spectra of the OPS are shown in FIG. 7A-B.

An inactivated strain of V. cholerae O100 serotype was provided by Nankai University, and LPS was extracted by a hot phenol-water method as reported previously. The strain was suspended in sterile water, and after multiple freezing and thawing cycles, a bacterial suspension and 90% phenol were mixed and shaken at 68° C. for 30 min. The mixture was cooled and centrifuged, and an aqueous phase was collected. An equal volume of sterile water was added to an organic phase and shaken at 68° C. for 30 min. The mixture was cooled and centrifuged again to separate an aqueous phase. The two aqueous phases were combined, dialyzed overnight with distilled water, and freeze-dried to obtain crude LPS. The crude LPS was further treated with DNase I, RNase A, and proteinase K in a Tris buffer solution (0.1 M, pH=8). Then the solution was heated at 100° C. for 10 min, and then cooled and centrifuged. A supernatant was extracted with water-saturated phenol. Following centrifugation, an aqueous phase was collected, dialyzed with distilled water, and freeze-dried to obtain purified LPS.

The LPS was de-lipidated with a 2% acetic acid aqueous solution at 100° C. until lipid A precipitated (3 h). Centrifugation (13,000 revolutions, 20 min) was carried out to remove precipitate, and the resulting product was purified using a G50 gel column to obtain OPS.

### Example 6

Effective titers of antibodies in rabbit sera were evaluated via ELISA, as shown in FIG. 8.

8 New Zealand rabbits (male, 1.8-2.2 kg, Wuxi Hengtai Experimental Animal Breeding Co., Ltd.) were randomly divided into a control group and an experimental group. Experimental group: 4 New Zealand rabbits were subcutaneously injected with a mixture of LPS of V. cholerae O100 and Freund's adjuvant at a ratio of 1:1 at different sites every 14 days (LPS: 0.4 mg/animal), and immunized three times (day 0, day 14, and day 28); and blood was collected from the ear vein of the rabbits. Control group: 4 New Zealand rabbits were subcutaneously injected with a mixture of PBS and Freund's adjuvant at a ratio of 1:1 at different sites every 14 days (day 0, day 14, and day 28). The anti-sera were aliquoted and stored at −80° C. Sera were collected on days 0, 7, 14, 28, and 35, and IgG antibodies in the sera were detected via enzyme-linked immunosorbent assay (ELISA). A P/N value represents a ratio of absorbance between the experimental group and the control group. When the P/N value of the immune group/control group is greater than or equal to 2.1, it is considered that the immune response of the IgG antibodies to the LPS of V. cholerae O100 serotype is positive.

### Specific Operations and Steps of ELISA

(1) Coating and washing: An ELISA plate was coated with antigens (20 μg/ml, 100 μl/well) at 4° C. for 24 h, and the plate was washed with PBST three times, and blotted dry on absorbent paper.

(2) Blocking and washing: A blocking solution (PBST containing 5% skim milk powder) was added to the coated ELISA plate (300 μl/well), and the plate was blocked overnight at 4° C., washed 3 times with PBST, and blotted dry.

(3) Addition of sera to be tested: Sera diluted with 1% BSA-PBS (at a dilution ratio of 1:12,800) were added to the ELISA plate (100 μl/well); a blank control (containing only 1% BSA-PBS) was also added to the ELISA plate; and the plate was incubated overnight at 4° C., washed 4 times, and blotted dry.

(4) Addition of ELISA secondary antibodies: Anti-rabbit HRP secondary antibodies diluted with 1% BSA-PBS (at a dilution ratio of 1:2,000) were added (100 μl/well), and the plate was incubated at 37° C. for 1 h, washed 4 times, and blotted dry.

(5) Color development: A TMB color solution was added (200 μl/well); the plate was incubated in the dark for 10 min, and then immediately quenched with 1 M diluted H₂SO₄ (50 μl/well); and the absorbance was read at 450 nm using an ELISA reader.

(6) Cleaning: After treatment of the sample, the sulfuric acid in the 96 well plate was neutralized with an ammonium bicarbonate solution.

The results are shown in FIG. 8, where 1-1, 1-2, 1-3, and 1-4 represent rabbit serum samples from four parallel experiments in the experimental group, respectively. The results indicated that after 7 days of immunization of rabbits, the P/N values began to increase significantly, and after 14 days, the P/N values of all samples were greater than 2.1, indicating that the immune response of the IgG antibodies to the LPS of V. cholerae O100 serotype was positive.

### Example 7

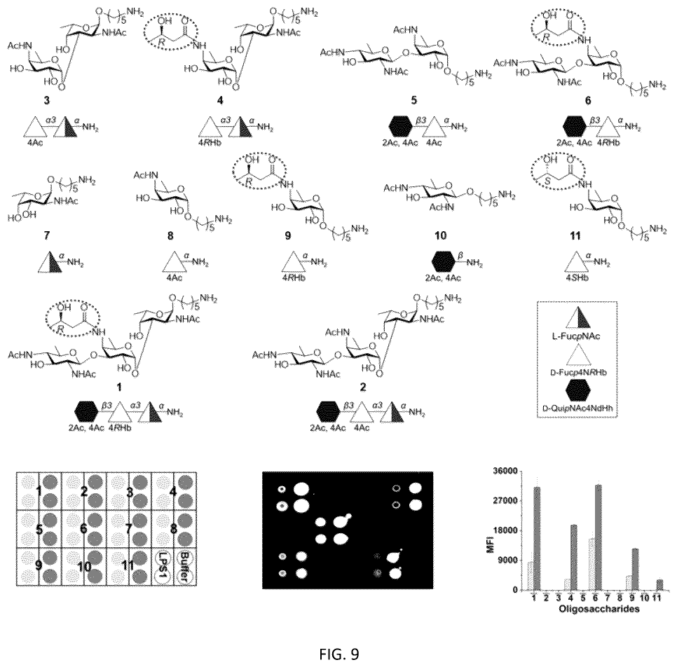

A specific saccharide antigen was screened by a glycan microarray, as shown in FIG. 9.

### Specific Experimental Operations and Steps:

Synthetic oligosaccharides and LPS were dissolved in a coupling buffer (50 mM sodium phosphate, pH=8.5) for printing onto a “CodeLink” slide (SurModics Co., Ltd.) using an RMA-Arrayer 96 (Rayme China). Then the slide was incubated overnight in a humidifying chamber at 26° C. and 55% humidity. The slide and the microarray were incubated in a quenching buffer (50 nM Na₂HPO₄, 100 nM ethanolamine) at 50° C. for 1 h. After being washed with distilled water and centrifuged, the quenched slide was blocked with 3% BSA (w/v) in PBS at room temperature for 1 h. The slide was washed with PBST (0.1% Tween in PBS) once and with PBS twice. After being centrifuged, the slide was placed in a culture chamber (ProPlate). Rabbit sera were diluted with 1% PBS-BSA (w/v) at a ratio of 1:200 and added to an incubation chamber. Each sample had at least four replicates. The microarray was incubated overnight in a dark and humid room at 4° C. Following washing 3 times with PBST, goat anti-rabbit IgG (Thermo) secondary antibodies were added per well, and incubated in a dark and humid room at 37° C. for 60 min, where the secondary antibodies were diluted with 1% PBS-BSA (w/v) at a ratio of 1:400. Then, the slide was rinsed with PBST three times, rinsed with water three times within 15 min, and centrifuged. Finally, the microarray was scanned using LuxScan 10K/B (CapitalBio Technology). Image analysis was carried out using GenePix Pro 7 software (Molecular Devices).

As shown in FIG. 9, compounds 1, 4, 6, 9, and 11 that contain 3-hydroxybutyryl have significant antigenicity, while compounds 2, 3, 5, 7, 8, and 10 that lack 3-hydroxybutyryl cannot be recognized by the antibodies, indicating that the 3-hydroxybutyryl plays a critical role in antibody recognition. The non-reducing end disaccharide 6 exhibits strong antibody recognition capacity, and is considered as the minimal antigenic epitope. These findings provide important reference for the development of glycoconjugate V. cholerae vaccine.

The examples provided above are not intended to limit the scope covered by the present disclosure, nor are the steps described to limit the execution order. Apparent improvements made to the present disclosure by those skilled in the art based on existing common knowledge also fall within the scope of protection defined in the claims of the present disclosure.

What is claimed is:

1. A specific saccharide fragment for development of Vibrio cholerae vaccines, having a structure of R₂—[U1]ₐ-[U2]—[U3]_{b}-O-Linker, the structures of U1, U2, and U3 being as follows: 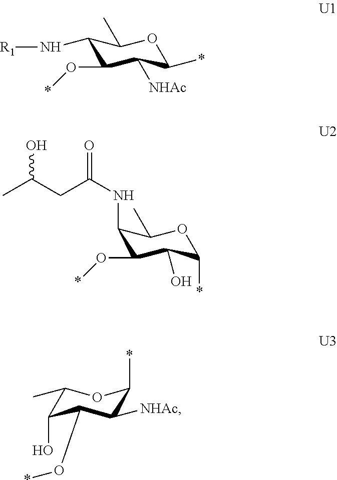
wherein a and b represent the quantities of U1 and U3, respectively, and a and b are 0 or 1, respectively;
R₁ represents one of 3,5-dihydroxyhexanoyl or acetyl groups;
R₂ represents H, or H-U3-, or H-U2-U3-, or H-U1-U2-U3-; and
Linker represents —(CH₂)ₙ—NH₂ or —(CH)ₙSH, wherein n=2-40.

2. The specific saccharide fragment according to claim 1, wherein a group at position 4 of U2 in the specific saccharide fragment is (R)-3-hydroxybutyrylamino or(S)-3-hydroxybutyrylamino.

3. The specific saccharide fragment according to claim 1, wherein the specific saccharide fragment is selected from: 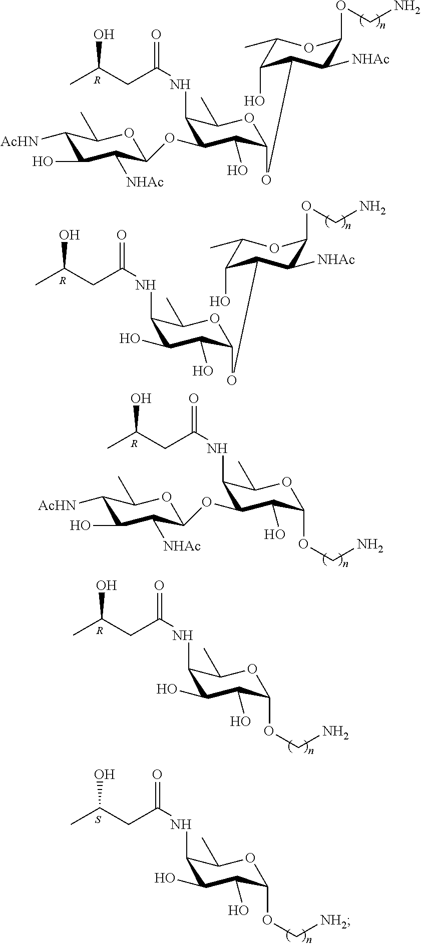
n=2−40.

4. A pharmaceutical composition, comprising the specific saccharide fragment according to claim 1 and pharmaceutical excipients.

5. A pharmaceutical composition, containing any one or a combination of more of the five types of specific saccharide fragments according to claim 3, and pharmaceutical excipients.

6. A glycan microarray, prepared by binding a Linker structure of the specific saccharide fragment according to claim 1 with the glycan microarray.

7. A Vibrio cholerae glycoprotein conjugate for vaccine development, obtained by conjugating a Linker structure of the specific saccharide fragment according to claim 1 with a protein.
